(19) United States
(12) Patent Application Publication
(10) Pub. No.: US 2023/0136423 A1
(43) Pub. Date: May 4, 2023

(54) NUCLEASE-MEDIATED PLASMID INTEGRATION

(71) Applicant: Inscripta, Inc., Boulder, CO (US)

(72) Inventors: Patrick WESTFALL, Boulder, CO (US); Catherine BARANOWSKI, Boulder, CO (US)

(73) Assignee: Inscripta, Inc., Boulder, CO (US)

(21) Appl. No.: 17/975,844

(22) Filed: Oct. 28, 2022

Related U.S. Application Data
(60) Provisional application No. 63/273,838, filed on Oct. 29, 2021.

(51) Int. Cl. C12N 15/10; C12N 9/22; C12N 15/11; C12N 15/72; C12N 1/20

(52) U.S. Cl. CPC C12N 15/1082; C12N 9/22; C12N 15/11; C12N 15/72; C12N 1/20; C12N 15/1065; C12N 2310/20; C12N 2800/80; C12N 2800/101

(57) ABSTRACT

The present disclosure relates to methods and compositions for nuclease-mediated plasmid integration into the genome of a population of live cells, as well as automated multi-module instruments for performing these methods and using these compositions.

### CROSS-REFERENCE TO RELATED APPLICATION

This application claims benefit of U.S. Provisional Application No. 63/273,838, filed Oct. 29, 2021, which is incorporated by reference herein in its entirety.

### FIELD OF THE INVENTION

The present disclosure relates to methods and compositions for nuclease-mediated plasmid integration into the genome of a population of live cells, as well as automated multi-module instruments for performing these methods and using these compositions.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

The ability to make precise, targeted changes to the genome of living cells has been a long-standing goal in biomedical research and development. In recent years, various genome editing tools have been explored for editing both simple and complex genomes, including the utilization of nucleic acid-guided nucleases. Such nucleases have enabled researchers to generate permanent genome edits in live cells, including insertions, deletions, integrations, and sequence substitutions.

Though offering great promise, current systems and methods utilizing nucleic acid-guided nucleases are not without their challenges. One such challenge is the efficient insertion of large fragments of DNA, e.g., 100 base pairs or more, into the genomes of live cells. Of course, it is desirable to achieve such insertions at the highest editing rates possible.

There is therefore a need in the art of nucleic acid-guided nuclease editing for improved methods, compositions, modules, and instruments for increasing the efficiency of editing large genomic fragments, and in particular, inserting payloads of 100 base pairs or more into a cellular genome. The present disclosure addresses this need.

### SUMMARY OF ILLUSTRATIVE EMBODIMENTS

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present disclosure relates to methods, compositions, and automated multi-module cell processing instruments for nucleic acid-guided nuclease editing to integrate plasmids into one or more target loci in a population of cells in a multiplexed manner. With the present compositions and methods, plasmid integration into one or more target loci is facilitated utilizing a nucleic-acid guided nuclease, which enables sequence-directed double stranded breaks in genomic and plasmid target sequences, upon which the plasmid sequence(s) may be inserted into the genomic DNA via homology-directed repair (HDR), non-homologous end joining (NHEJ), or by recombination pathways. An advantage of the present methods and compositions is that they enable one to leverage CRISPR-type nucleic acid-guided nuclease genome-wide targeted editing to reliably integrate, i.e., embed, a plasmid in the cellular genome, thus facilitating insertion or substitution of large DNA sequences (e.g., >100 bp), including sequences of one or more genes of a metabolic pathway.

In some aspects, there is provided an editing cassette for performing nucleic acid-guided nuclease genome editing to integrate plasmids. In some aspects, the editing cassette comprises a gRNA having homology to a target sequence (i.e., target loci or target region) in a cell genome, and/or a donor nucleic acid sequence, e.g., a donor DNA sequence. In some aspects, the editing cassette is agnostic to the order of the gRNA and donor nucleic acid sequence. In some aspects, the gRNA and donor DNA sequence are under the control of a promoter at the 5′ end of the editing cassette.

In some aspects, a region of complementarity between the gRNA and a target sequence is between 4-120 nucleotides in length, or between 5-80 nucleotides in length, or between 6-60 nucleotides in length. The gRNA may be designed to bind with the template or non-template strand of double stranded DNA.

In some aspects, a target sequence in a cell genome comprises a neutral integration site, or “safe spot,” that facilitates stable integration of a plasmid and/or supports high gene expression without significant impact on growth rate of the cell. In specific aspects, neutral integration sites are selected based on inspection of a host GFP fusion localization database. In specific aspects, a neutral integration site is disposed centrally in a large intergenic region to reduce potential of plasmid integration from adversely affecting genes neighboring the integrated plasmid. In specific aspects, where a plurality of plasmid integrations and/or sequence insertions are performed, the plasmids and/or sequences are embedded into one or more clustered neutral integration sites.

In specific aspects, the editing cassette further comprises a barcode sequence and/or an amplification priming site at the 3′ end of the editing cassette. In specific aspects, the editing cassette further comprises a melting temperature booster sequence at the 5′ end of the editing cassette, which is a short protective DNA sequence. In addition, in specific aspects, the editing cassette comprises regions of homology to a vector for gap-repair insertion of the editing cassette into the vector, such as an editing plasmid or engine vector.

In some aspects, the editing cassette comprises one or more gRNAs.

In some aspects, the donor DNA sequence is from about 10 bp to about 100 Kb in length, and in specific aspects, the donor DNA sequence is from about 1 Kb to about 50 Kb in length, or from about 5 Kb to about 25 Kb in length, or from about 10 Kb to about 20 Kb in length, or from about 12 Kb to about 15 Kb in length. In specific aspects, the donor DNA sequence is from about 15 bp to about 500 bp in length, or from about 20 bp to about 400 bp in length, or from about 50 bp to about 250 bp in length, or from about 100 bp to about 200 bp in length. In specific aspects, the donor DNA sequence is from about 500 bp to about 5 Kb in length, or from about 1 Kb to about 4 Kb in length, or from about 2 Kb to about 3 Kb in length.

In some aspects, the donor DNA sequence comprises one or more genes. In specific aspects, the one or more genes includes a plurality of genes for an endogenous or heterologous metabolic pathway. In some aspects, where the donor DNA sequence comprises a plurality of genes, each of the genes may be separated from other genes by a linker or spacer. In some aspects, the one or more genes are driven by bi-directional promoters.

In some aspects, there is provided an editing plasmid (e.g., a donor plasmid) for performing nucleic acid-guided nuclease genome editing, the editing plasmid to be integrated into the cell genome during editing. In some aspects, the editing plasmid comprises an editing cassette, which comprises a gRNA and/or a donor nucleic acid sequence. In some aspects, a gRNA and/or donor nucleic acid is integrated into an editing plasmid backbone prior to assembly with an editing cassette. In some aspects, the editing plasmid further includes an origin of replication and a selectable marker component, e.g., an antibiotic resistance gene or a fluorescent protein gene. In some aspects, the editing plasmid further comprises a barcode sequence, which may be different from a barcode sequence of the editing cassette. In some aspects, the editing plasmid further includes a nuclease. In some aspects, the editing plasmid further includes one or more promoters positioned to drive transcription of the gRNAs, the one or more genes, the selectable marker component, and/or the nuclease. The one or more promoters may be constitutive or inducible.

In some aspects, the editing plasmid comprises regions of homology to a target locus of a cell genome, or HDR sequences, for HDR-mediated insertion or substitution of the editing plasmid into the cell genome.

In some aspects, the editing plasmid comprises one or more editing cassettes.

In specific aspects, the editing plasmid is a linear plasmid. In specific aspects, the editing plasmid is a circular plasmid.

In specific aspects, the editing plasmid is a self-cutting plasmid and may further comprise a self-targeting sequence having homology to a gRNA of an editing cassette, as well as a protospacer adjacent motif (PAM) site, to induce a double-stranded break by a nuclease for integration of the plasmid into a target loci in a cell genome during editing.

In some aspects, the editing plasmid or editing cassette further comprises a “landing pad” sequence, or a sequence of nucleotides comprising an enzyme recognition sequence, such as a recombinase, integrase, nuclease, or meganuclease recognition sequence. The landing pad can be leveraged to insert additional, large donor nucleic acid sequences (i.e., large DNA payloads), including additional editing plasmids, in recursive editing operations. For example, after an initial editing operation wherein a plasmid (or editing cassette) comprising a landing pad is integrated into a genome, a subsequent editing operation utilizing a vector comprising an additional donor DNA sequence and a coding sequence for, e.g., a recombinase, integrase, nuclease, or meganuclease may be performed. Inducing expression of the coded enzyme may facilitate insertion of the additional donor DNA sequence into the previously-integrated landing pad.

In specific aspects, the recombinase is a cyclization recombination enzyme (Cre) and the landing pad and/or additional donor DNA sequence comprise lox recombination sites. In specific aspects, the recombinase is a flippase enzyme and the landing pad and/or additional donor DNA sequence comprise flippase recognition targets (FRTs).

In specific aspects, the vector carrying the additional donor DNA sequence comprises a coding sequence for a meganuclease, the landing pads comprise a recognition sequence for the meganuclease, and the additional donor DNA sequence comprises homologous sequences flanking the DNA payload. In some aspects, the meganuclease belongs to the LAGLIDADG family of nucleases, and in some aspects, the meganuclease is I-SceI; the meganuclease is I-CreI; or the meganuclease is I-DmoI.

In some aspects, the vector carrying the additional donor DNA sequence comprises the coding sequence of the recombinase or meganuclease under the control of an inducible promoter. In some aspects, the inducible promoter is a pL promoter or a pBAD promoter.

In some aspects, the additional donor DNA sequence is from about 100 bp to about 100 Kb in length, and in specific aspects, the sequence is from about 1 Kb to about 50 Kb in length, or from about 5 Kb to about 25 Kb in length, or from about 10 Kb to about 20 Kb in length, or from about 12 Kb to about 15 Kb in length. In specific aspects, the additional donor DNA sequence is from about 15 bp to about 500 bp in length, or from about 20 bp to about 400 bp in length, or from about 50 bp to about 250 bp in length, or from about 100 bp to about 200 bp in length. In specific aspects, additional the donor DNA sequence is from about 500 bp to about 5 Kb in length, or from about 1 Kb to about 4 Kb in length, or from about 2 Kb to about 3 Kb in length.

In some aspects, the additional donor DNA sequence comprises one or more genes. In specific aspects, the one or more genes includes a plurality of genes for an endogenous or heterologous metabolic pathway. In some aspects, where the additional donor DNA sequence comprises a plurality of genes, each of the genes may be separated from other genes by a linker or spacer.

In some aspects, the vector carrying the additional donor DNA sequence further comprises an origin of replication and a selectable marker.

In specific aspects, the vector carrying the additional donor DNA sequence is a linear plasmid. In specific aspects, the vector carrying the additional donor DNA sequence is a circular plasmid.

In some aspects, the vector carrying the additional donor DNA sequence is a second editing plasmid. In specific aspects, the second editing plasmid further comprises a coding sequence for a recombinase, integrase, nuclease, or meganuclease.

In some aspects, there is provided an engine vector for performing nucleic acid-guided nuclease genome editing to integrate plasmids. In specific aspects, the engine vector comprises a nuclease, an optional selectable marker, e.g., an antibiotic resistance gene, and an optional barcode sequence. In some aspects, the engine vector further comprises a promoter positioned to drive transcription of the nuclease and/or the selectable marker. The promoter may be constitutive or inducible.

In some aspects, there is provided a combined engine/editing plasmid for performing nucleic acid-guided nuclease genome editing to integrate plasmids. In specific aspects, the combined engine/editing plasmid comprises one or more gRNAs and a donor DNA sequence. In specific aspects, the combined engine/editing plasmid further comprises a nuclease, a selectable marker, and a barcode sequence. In some aspects, the combined engine/editing plasmid further includes one or more promoters positioned to drive transcription of the gRNA, the donor DNA sequence, the selectable marker component, and/or the nuclease. The one or more promoters may be constitutive or inducible.

In some aspects, a nuclease may be introduced into cells using a DNA molecule coding for the nuclease separately or covalently-linked to an editing plasmid comprising one or more gRNAs, or the nuclease may be introduced into cells using a DNA molecule coding for the nuclease separately or covalently-linked to an engine vector, or the nuclease may be introduced separately in polypeptide/protein form or as part of a complex, or the nuclease may be introduced into cells using an mRNA coding for the nuclease. In addition to the nuclease, the editing plasmid comprising one or more gRNAs is utilized.

In some aspects, the nuclease includes a MAD-series nuclease or a variant (e.g., orthologue) thereof. In specific aspects, the nuclease includes MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, MAD20, MAD2001, MAD2007, MAD2008, MAD2009, MAD2011, MAD2017, MAD2019, MAD297, MAD298, MAD299, or other MAD-series nucleases, variants thereof, and/or combinations thereof.

In some aspects, a nickase is utilized to mediate plasmid integration. In specific aspects, the one or more nickases include MAD7 nickase, MAD2001 nickase, MAD2007 nickase, MAD2008 nickase, MAD2009 nickase, MAD2011 nickase, MAD2017 nickase, MAD2019 nickase, MAD297 nickase, MAD298 nickase, MAD299 nickase, or other MAD-series nickases, variants thereof, and/or combinations thereof.

In some aspects, the nuclease includes C2c1, C2c2, C2c3, Cas1, Cas1B, Cas2, Cas3, Cas4, Cas5, Cash, Cas7, Cas8, Cas9 (also known as Csn1 and Csx12), Cas10, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx100, Csx16, CsaX, Csx3, Csx1, Csx15, Csf1, Csf2, Csf3, Csf4, variants thereof, and/or combinations thereof.

In some aspects, there is provided a library of vector or plasmid backbones and/or a library of editing cassettes to be transformed into cells. In some aspects, one or more editing cassettes in the library of editing cassettes each comprise a different gRNA targeting a different target locus within the cell genomes, and/or a different donor DNA sequence. In some aspects, the utilization of a library of editing cassettes, and in certain cases, a library of vector or plasmid backbones, enables combinatorial or multiplex editing in the cells.

The present disclosure includes methods of using for nucleic acid-guided nuclease editing in cell populations, e.g., bacterial and fungal cells. The cells that can be used with the methods of the present disclosure include any prokaryotic, archaeal, or eukaryotic cells. For example, prokaryotic cells for use with the present illustrative embodiments can include gram-positive bacterial cells, e.g., Bacillus subtilis, or gram-negative bacterial cells, e.g., Escherichia coli cells. Eukaryotic cells for use with the automated multi-module cell processing instruments of the illustrative embodiments include any plant cells and any animal cells, e.g., fungal cells (including yeast), insect cells, amphibian cells, and the like. In specific aspects, methods of the present disclosure are used with common research microbial species such as E. coli or Saccharomyces cerevisiae. Other model organisms that can be used with the methods and compositions of the present disclosure include Streptomyces spp., Pseudomonas spp., Corynebacterium spp., Bacillus spp., Aspergillus spp., Vibrio spp., Yarrowia lypolytica, and Pichia pastoris.

In some aspects, the present disclosure provides methods of inserting one or more exogenous genes into a live cell, i.e., methods of performing gene knockin (KI).

In some aspects, the present disclosure provides methods of inserting one or more metabolic pathway genes into cells for biosynthesis of one or more desired bioproducts. In specific aspects, the insertion of one or more metabolic pathway genes comprises insertion of a plurality of genes involved in a metabolic pathway. In specific aspects, the insertion of one or more metabolic pathway genes enables the biosynthesis of the one or more desired bioproducts by the cells. In such aspects, the cells used with the methods of the present disclosure include microbial cells, which may serve as mini “factories” to produce a desired bioproduct. Accordingly, the present disclosure provides efficient methods for metabolic engineering.

In some aspects, the microbes used with the methods of the present disclosure include bacterial cells. In some aspects, the microbes used with the methods of the present disclosure include fungal cells.

In some aspects, the one or more genes inserted by the methods of the present disclosure include genes heterologously introduced from another organism or species.

In some aspects, automated methods are used for nucleic acid-guided nuclease editing in multiple cells to insert one or more metabolic pathway genes for biosynthesis of one or more desired bioproducts, the methods being performed in automated multi-module cell processing instruments. The automated methods carried out using the automated multi-module cell processing instruments described herein can be used with a variety of nucleic acid-guided genome editing techniques, and can be used with or without use of one or more selectable markers.

The present disclosure thus provides, in selected embodiments, modules, instruments, and systems for automated multi-module cell processing for nuclease-mediated genome editing in multiple cells. Automated systems for cell processing that may be used for can be found, e.g., in U.S. Pat. Nos. 10,253,316; 10,329,559; 10,323,242; 10,421,959; 10,465,185; 10,519,437; 10,584,333; 10,584,334; 10,647,982; 10,689,645; 10,738,301; and 10,738,663.

In some aspects, the automated multi-module cell processing instruments of the present disclosure are designed for nucleic acid-guided genome editing, such as recursive genome editing, e.g., sequentially introducing multiple edits into genomes inside one or more cells of a cell population through two or more editing operations within the instruments.

In some aspects, the methods of the present disclosure facilitate a plasmid integration frequency of greater than 10% in clonal cell lines, such as a plasmid integration frequency of greater than 15%, such as a plasmid integration frequency of greater than 20%, such as a plasmid integration frequency of greater than 25%, such as a plasmid integration frequency of greater than 30%, such as a plasmid integration frequency of greater than 35%, such as a plasmid integration frequency of greater than 40%, such as a plasmid integration frequency of greater than 45%, such as a plasmid integration frequency of greater than 50%, such as a plasmid integration frequency of greater than 55%, such as a plasmid integration frequency of greater than 60%, such as a plasmid integration frequency of greater than 65%, such as a plasmid integration frequency of greater than 70%, such as a plasmid integration frequency of greater than 75%, such as a plasmid integration frequency of greater than 80%, such as a plasmid integration frequency of greater than 85%, such as a plasmid integration frequency of greater than 90%.

These aspects and other features and advantages of the invention are described below in more detail.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIG. 1A is a simplified block diagram of an exemplary method for performing nucleic acid-guided nuclease editing in a population of cells to embed plasmids into target genetic loci of the cells. FIG. 1B is a simplified depiction of plasmid integration as described in FIG. 1A. FIG. 1C is a simplified depiction of an exemplary operation described in FIG. 1A.

FIGS. 2A, 2B, and 2C depict an automated multi-module instrument and components thereof with which to practice the nucleic acid-guided nuclease editing to integrate plasmids, as taught herein.

FIG. 3A depicts one embodiment of a rotating growth vial for use with the cell growth module described herein. FIG. 3B illustrates a perspective view of one embodiment of a rotating growth vial in a cell growth module. FIG. 3C depicts a cut-away view of the cell growth module from FIG. 3B. FIG. 3D illustrates the cell growth module of FIG. 3B coupled to LED, detector, and temperature regulating components.

FIG. 4A is a model of tangential flow filtration used in the TFF device presented herein. FIG. 4B depicts a top view of a lower member of one embodiment of an exemplary TFF device. FIG. 4C depicts a top-down view of the reservoir assemblies 450 shown in FIG. 4B. FIG. 4D depicts a cover 444 for reservoir assembly 450 shown in FIG. 4B. FIG. 4E depicts a gasket 445 that in operation is disposed on cover 444 of reservoir assemblies 450 shown in FIG. 4B.

FIG. 5A shows a flow-through electroporation device exemplary (here, there are six such devices co-joined). FIG. 5B is a top view of one embodiment of an exemplary flow-through electroporation device. FIG. 5C depicts a top view of a cross section of the electroporation device of FIG. 5C. FIG. 5D is a side view cross section of a lower portion of the electroporation devices of FIGS. 5C and 5D. Additional details of the flow-through electroporation devices are illustrated in FIGS. 5E and 5F.

FIGS. 6A and 6B depict the structure and components of one embodiment of a reagent cartridge. FIG. 6C is a top perspective view of a solid wall isolation, incubation and normalization (SWIIN) module with the retentate and perforated members in partial cross section. FIG. 6D is a side perspective view of an assembled SWIIIN module 650, including, from right to left, reservoir gasket 658 disposed upon integrated reservoir cover 678 (not seen) of retentate member 604. FIG. 6E depicts the embodiment of the SWIIN module in FIGS. 6B-6D further comprising a heat management system including a heater and a heated cover.

FIG. 7 is a simplified block diagram of an embodiment of an exemplary automated multi-module cell processing instrument.

### DETAILED DESCRIPTION

All of the functionalities described in connection with one embodiment of the methods, devices or instruments described herein are intended to be applicable to the additional embodiments of the methods, devices and instruments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

The practice of the techniques described herein may employ, unless otherwise indicated, conventional techniques and descriptions of molecular biology (including recombinant techniques), cell biology, biochemistry, and genetic engineering technology, which are within the skill of those who practice in the art. Such conventional techniques and descriptions can be found in standard laboratory manuals such as Green and Sambrook, Molecular Cloning: A Laboratory Manual. 4th, ed., Cold Spring Harbor Laboratory Press, Cold Spring Harbor, N.Y., (2014); Current Protocols in Molecular Biology, Ausubel, et al. eds., (2017); Neumann, et al., Electroporation and Electrofusion in Cell Biology, Plenum Press, New York, 1989; and Chang, et al., Guide to Electroporation and Electrofusion, Academic Press, California (1992), all of which are herein incorporated in their entirety by reference for all purposes. Nucleic acid-guided nuclease techniques can be found in, e.g., Genome Editing and Engineering from TALENs and CRISPRs to Molecular Surgery, Appasani and Church (2018); and CRISPR: Methods and Protocols, Lindgren and Charpentier (2015); both of which are herein incorporated in their entirety by reference for all purposes.

Note that as used herein and in the appended claims, the singular forms “a,” “an,” and “the” include plural referents unless the context clearly dictates otherwise. Thus, for example, reference to “a cell” refers to one or more cells, and reference to “the system” includes reference to equivalent steps, methods and devices known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Unless defined otherwise, all technical and scientific terms used herein have the same meaning as commonly understood by one of ordinary skill in the art to which this invention belongs. All publications mentioned herein are incorporated by reference for all purposes, including but not limited to describing and disclosing devices, formulations and methodologies that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in smaller ranges, and are also encompassed within the invention, subject to any specifically excluded limit in the stated range. Where the stated range includes one or both of the limits, ranges excluding either or both of those included limits are also included in the invention.

In the following description, numerous specific details are set forth to provide a more thorough understanding of the present invention. However, it will be apparent to one of skill in the art that the present invention may be practiced without one or more of these specific details. In other instances, features and procedures well known to those skilled in the art have not been described in order to avoid obscuring the invention. The terms used herein are intended to have the plain and ordinary meaning as understood by those of ordinary skill in the art.

The term “amino acid” refers to naturally occurring and synthetic amino acids, as well as amino acid analogs that function similarly to naturally occurring amino acids.

The terms “cassette,” “expression cassette,” “editing cassette,” “CREATE cassette,” “CREATE editing cassette,” “CREATE fusion editing cassette,” or “CFE editing cassette” refer to a nucleic acid molecule comprising a coding sequence for transcription of a guide nucleic acid or gRNA.

The term “complementary” as used herein refers to Watson-Crick base pairing between nucleotides and specifically refers to nucleotides hydrogen-bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-TCGA-5′ is 100% complementary to a region of the nucleotide sequence 5′-TTAGCTGG-3′.

The term DNA “control sequences” refers collectively to promoter sequences, polyadenylation signals, transcription termination sequences, upstream regulatory domains, origins of replication, internal ribosome entry sites, nuclear localization sequences, enhancers, and the like, which collectively provide for the replication, transcription and translation of a coding sequence in a recipient cell. Not all of these types of control sequences need to be present so long as a selected coding sequence is capable of being replicated, transcribed and—for some components—translated in an appropriate host cell.

The term “gene” refers to a segment of DNA involved in producing a polypeptide chain. It may include regions preceding and following a coding region (leader and trailer, respectively), as well as intervening sequences (introns) between individual coding segments (exons).

The terms “guide nucleic acid” or “guide RNA” or “gRNA” refer to a polynucleotide comprising 1) a guide sequence capable of hybridizing to a genomic target locus, and 2) a scaffold sequence capable of interacting or complexing with a nucleic acid-guided nuclease.

The term “heterologous” refers to the relationship between two or more nucleic acids or protein sequences from different sources, or the relationship between a protein (or nucleic acid) and a host cell from different sources. For example, if the combination of a nucleic acid and a host cell is usually not naturally occurring, the nucleic acid is heterologous to the host cell. A particular sequence is “heterologous” to the cell or organism into which it is inserted.

“Homology” or “identity” or “similarity” refers to sequence similarity between two peptides or, more often in the context of the present disclosure, between two nucleic acid molecules. The term “homologous region” or “homology arm” refers to a region on a donor DNA with a certain degree of homology with a target genomic DNA sequence. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

The terms “intermediate compound” and “intermediates” refer to a product of a synthesis pathway that is not the terminal product, but which is useful for the production of the final intended product. The term “naturally occurring” when used in reference to a bioproduct refers to a chemical compound or substance produced by a living organism. In the broadest sense, bioproducts include any substance produced by life, including substrates, enzymes, cofactors, and terminal products (e.g. final intended pesticides) and pathway intermediates of terminal products. The term also encompasses complex extracts and isolated compounds derived from those extracts. In the broadest sense, a chemical or product that is “naturally occurring” includes any substance or combination of substances produced by life. In addition, the term is intended to encompass a substance that forms the structural basis for commercial bioproducts, such as an intermediary product.

The term “landing pad” refers to a sequence of nucleotides inserted into a genome or episome of a cell via CRISPR editing that comprises an enzyme recognition sequence.

The term “meganuclease” refers to an endodeoxyribonuclease characterized by a large recognition site (double-stranded DNA sequences of 12 to 40 base pairs) and as a result the recognition site generally occurs only once, if at all, in any given genome.

The term “nucleic acid” or “polynucleotide” refers to deoxyribonucleic acids (DNA) or ribonucleic acids (RNA) and polymers thereof in either single- or double-stranded form. Unless otherwise indicated, the terms encompass nucleic acids containing known analogues or natural nucleotides that have similar binding properties as the reference nucleic acid and are metabolized in a manner similar to naturally occurring nucleotides. Unless otherwise indicated, in addition to the sequence specifically stated, a particular nucleic acid sequence also implicitly encompasses conservatively modified variants thereof (e.g., degenerate codon substitutions), alleles, orthologues, SNPs, and complementary sequences. The term nucleic acid is used interchangeably with gene, cDNA, and mRNA encoded by a gene.

The term “nucleic acid-guided editing components” refers to one, some, or all of a nucleic acid-guided nuclease or nickase fusion enzyme, a guide nucleic acid and a repair template and/or donor nucleic acid.

“Operably linked” refers to an arrangement of elements where the components so described are configured so as to perform their usual function. Thus, control sequences operably linked to a coding sequence are capable of effecting the transcription, and in some cases, the translation, of a coding sequence. The control sequences need not be contiguous with the coding sequence so long as they function to direct the expression of the coding sequence. Thus, for example, intervening untranslated yet transcribed sequences can be present between a promoter sequence and the coding sequence and the promoter sequence can still be considered “operably linked” to the coding sequence. In fact, such sequences need not reside on the same contiguous DNA molecule (i.e. chromosome) and may still have interactions resulting in altered regulation.

The term “PAM mutation” refers to one or more edits to a target sequence that removes, mutates, or otherwise renders inactive a PAM or spacer region in the target sequence.

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA transcribed by any class of any RNA polymerase I, II or III. Promoters may be constitutive or inducible.

As used herein, the terms “protein,” “peptide,” and “polypeptide” are used interchangeably herein and refer to a polymer of amino acid residues. Proteins may or may not be made up entirely of amino acids.

“Recognition sequences” are particular sequences of nucleotides that a protein, DNA, or RNA molecule, or combinations thereof (such as, but not limited to, a restriction endonuclease, a modification methylase or a recombinase) recognizes and binds. For example, a recognition sequence for Cre recombinase is a 34 base pair sequence containing two 13 base pair inverted repeats (serving as the recombinase binding sites) flanking an 8 base pair core and designated loxP (see, e.g., Sauer, Current Opinion in Biotechnology, 5:521-527 (1994)). Other examples of recognition sequences include, but are not limited to, attB and attP, attR and attL and others that are recognized by the recombinase enzyme bacteriophage Lambda Integrase. The recombination site designated attB is an approximately 33 base pair sequence containing two 9 base pair core-type Int binding sites and a 7 base pair overlap region; attP is an approximately 240 base pair sequence containing core-type Int binding sites and arm-type Int binding sites as well as sites for auxiliary proteins IHF, FIS, and Xis (see, e.g., Landy, Current Opinion in Biotechnology, 3:699-7071 (1993)).

A “recombinase” is an enzyme that catalyzes the exchange of DNA segments at specific recombination sites. An “integrase” refers to a recombinase that is usually derived from viruses or transposons, as well as perhaps ancient viruses. “Recombination proteins” include excisive proteins, integrative proteins, enzymes, co-factors and associated proteins that are involved in recombination reactions using one or more recombination sites (again see, e.g., Landy, Current Opinion in Biotechnology, 3:699-707 (1993)). The recombination proteins used in the methods herein can be delivered to a cell via an editing cassette on an appropriate vector, such as a plasmid or viral vector. In other embodiments, recombination proteins can be delivered to a cell in protein form in the same reaction mixture used to deliver the desired nucleic acid(s). In yet other embodiments, the recombinase could also be encoded in the cell and expressed upon demand using a tightly controlled inducible promoter.

As used herein, the terms “repair template” or “homology arm” refer to 1) nucleic acid that is designed to facilitate introduction of a DNA sequence modification (insertion, deletion, substitution) into a locus by homologous recombination using nucleic acid-guided nucleases, or 2) a nucleic acid that serves as a template (including a desired edit) to be incorporated into target DNA by reverse transcriptase in a CREATE fusion editing (CFE) system. For homology-directed repair, a repair template or homology arm may have sufficient homology to the regions flanking the “cut site” or the site to be edited in the genomic target sequence. For template-directed repair, the repair template or homology arm has homology to the genomic target sequence except at the position of the desired edit although synonymous edits may be present in the homologous (e.g., non-edit) regions. The length of the repair template(s) or homology arm(s) will depend on, e.g., the type and size of the modification being made. In many instances and preferably, the repair template will have two regions of sequence homology (e.g., two homology arms) complementary to the genomic target locus flanking the locus of the desired edit in the genomic target locus. Typically, an “edit region” or “edit locus” or “DNA sequence modification” region—the nucleic acid modification that one desires to be introduced into a genome target locus in a cell (e.g., the desired edit)—will be located between two regions of homology. The DNA sequence modification may change one or more bases of the target genomic DNA sequence at one specific site or multiple specific sites. A change may include changing 1, 2, 3, 4, 5, 10, 15, 20, 25, 30, 35, 40, 50, 75, 100, 150, 200, 300, 400, or 500 or more base pairs of the target sequence. A deletion or insertion may be a deletion or insertion of 1, 2, 3, 4, 5, 10, 15, 20, 25, 30, 40, 50, 75, 100, 150, 200, 300, 400, or 500 or more base pairs of the target sequence.

As used herein the term “selectable marker” refers to a gene introduced into a cell, which confers a trait suitable for artificial selection. General use selectable markers are well-known to those of ordinary skill in the art. For example, selectable markers can use means that deplete a cell population to enrich for editing or gene regulation, and include ampicillin/carbenicillin, kanamycin, chloramphenicol, nourseothricin N-acetyl transferase, erythromycin, tetracycline, gentamicin, bleomycin, streptomycin, puromycin, hygromycin, blasticidin, and G418 or other selectable markers may be employed. In addition, selectable markers include physical markers that confer a phenotype that can be utilized for physical or computations cell enrichment, e.g., optical selectable markers such as fluorescent proteins (e.g., green fluorescent protein, blue fluorescent protein) and cell surface handles.

The term “specifically binds” as used herein includes an interaction between two molecules, e.g., an engineered peptide antigen and a binding target, with a binding affinity represented by a dissociation constant of about 10⁻⁷M, about 10⁻⁸M, about 10⁻⁹ M, about 10⁻¹⁰ M, about 10⁻¹¹M, about 10⁻¹² M about 10⁻¹³ M, about 10⁻¹⁴ M or about 10⁻¹⁵ M.

The terms “target genomic DNA sequence”, “cellular target sequence”, “target sequence”, or “target locus,” for purposes of the present disclosure, refer to any locus in vitro or in vivo, or in a nucleic acid (e.g., genome or episome) of a cell or population of cells, in which a change of at least one nucleotide is desired using a nucleic acid-guided nuclease editing system. The target sequence can be a genomic locus or extrachromosomal locus.

The terms “transformation”, “transfection” and “transduction” are used interchangeably herein to refer to the process of introducing exogenous DNA into cells.

The term “variant” may refer to a polypeptide or polynucleotide that differs from a reference polypeptide or polynucleotide but retains essential properties. A typical variant of a polypeptide differs in amino acid sequence from another reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A variant of a polypeptide may be a conservatively modified variant. A substituted or inserted amino acid residue may or may not be one encoded by the genetic code (e.g., a non-natural amino acid). A variant of a polypeptide may be naturally occurring, such as an allelic variant, or it may be a variant that is not known to occur naturally.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, synthetic chromosomes, and the like. In the present disclosure, the term “combined engine/editing vector” may include a coding sequence for a nuclease and an editing cassette comprising a gRNA sequence to be transcribed. In other embodiments, however, two vectors—a recombinant engine vector comprising the coding sequence for a nuclease, and an editing cassette, comprising the gRNA sequence to be transcribed—may be used.

Nuclease-Directed Genome Editing with Plasmid Integration, Generally

The compositions, methods, modules and instruments described herein are employed to allow one to perform nucleic acid nuclease-directed genome editing (i.e., CRISPR editing) to introduce desired edits to a population of live cells. Specifically, the compositions, methods, modules and integrated instruments presented herein facilitate editing nucleotide sequences in a population of cells in a multiplexed and targeted manner, including insertions of donor plasmids, i.e., editing plasmids, comprising large donor DNA sequences or payloads (e.g., >100 bp, >500 bp, >1 Kb, >2 Kb, >3 Kb, >4 Kb, >5 Kb, >10 Kb, >15 Kb, >20 Kb, >25 Kb, >50 Kb, >100 Kb). An advantage of the present methods and compositions is that they allow one to leverage CRISPR-type nucleic acid-guided nuclease genome-wide targeted editing to insert entire plasmids in a cellular genome, which may comprise large DNA payloads, such as one or more genes of a metabolic pathway.

In CRISPR editing, a nucleic acid-guided nuclease complexed with an appropriate synthetic guide nucleic acid in a cell can cut the genome of the cell at a desired location. The guide nucleic acid helps the nucleic acid-guided nuclease recognize and cut the DNA at a specific target sequence. By manipulating the nucleotide sequence of the guide nucleic acid, the nucleic acid-guided nuclease may be programmed to target any DNA sequence for cleavage as long as an appropriate protospacer adjacent motif (PAM) is nearby. In certain aspects, the nucleic acid-guided nuclease editing system may use two separate guide nucleic acid molecules that combine to function as a guide nucleic acid, e.g., a CRISPR RNA (crRNA) and trans-activating CRISPR RNA (tracrRNA). In other aspects and preferably, the guide nucleic acid is a single guide nucleic acid construct that includes both 1) a guide sequence capable of hybridizing to a genomic target locus, and 2) a scaffold sequence capable of interacting or complexing with a nucleic acid-guided nuclease.

A guide nucleic acid (e.g., gRNA) complexes with a compatible nucleic acid-guided nuclease and can then hybridize with a target sequence, thereby directing the nuclease to the target sequence. A guide nucleic acid can be DNA or RNA; alternatively, a guide nucleic acid may comprise both DNA and RNA. In some embodiments, a guide nucleic acid may comprise modified or non-naturally occurring nucleotides. In cases where the guide nucleic acid comprises RNA, the gRNA may be encoded by a DNA sequence on a polynucleotide molecule such as a plasmid, linear construct, or the coding sequence may and preferably does reside within an editing cassette. Methods and compositions for designing and synthesizing editing cassettes and libraries of editing cassettes are described in U.S. Pat. Nos. 10,240,167; 10,266,849; 9,982,278; 10,351,877; 10,364,442; 10,435,715; 10,465,207; 10,669,559; 10,711,284; 10,731,180; and 11,078,498; all of which are incorporated by reference herein.

A guide nucleic acid comprises a guide sequence, where the guide sequence is a polynucleotide sequence having sufficient complementarity with a target sequence to hybridize with the target sequence and direct sequence-specific binding of a complexed nucleic acid-guided nuclease to the target sequence. The degree of complementarity between a guide sequence and the corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some embodiments, a guide sequence is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20 nucleotides in length. Preferably the guide sequence is 10-30 or 15-20 nucleotides long, or 15, 16, 17, 18, 19, or 20 nucleotides in length.

To facilitate plasmid integration at a target sequence, thus generating an edit therein, the gRNA/nuclease complex binds to the target sequence as determined by the guide RNA, and the nuclease recognizes a protospacer adjacent motif (PAM) sequence adjacent to the target sequence. The target sequence can be any polynucleotide endogenous or exogenous to the cell, or in vitro. For example, the target sequence can be a polynucleotide residing in the nucleus of the cell. A target sequence can be a sequence encoding a gene product (e.g., a protein) or a non-coding sequence (e.g., a regulatory polynucleotide, an intron, a PAM, a control sequence, or “junk” DNA).

In certain embodiments, the guide nucleic acid may be part of an editing cassette that encodes a desired donor nucleic acid for insertion into the cellular target sequence, and/or one or more homology arms. Alternatively, the guide nucleic acid may not be part of the editing cassette and instead may be encoded on the vector backbone, such as an editing plasmid backbone. For example, a sequence coding for a guide nucleic acid can be assembled or inserted into an editing plasmid backbone first, followed by insertion of the donor nucleic acid sequence in, e.g., an editing cassette. In other cases, the donor nucleic acid sequence in, e.g., an editing cassette can be inserted or assembled into an editing plasmid backbone first, followed by insertion of the sequence coding for the guide nucleic acid. In certain embodiments, the sequence encoding the guide nucleic acid and the donor nucleic acid sequence are located together in a rationally-designed editing cassette and are simultaneously inserted or assembled via gap repair into a linear plasmid or backbone to create an editing plasmid.

The target sequence is associated with a proto-spacer mutation (PAM), which is a short nucleotide sequence recognized by the gRNA/nuclease complex. The precise preferred PAM sequence and length requirements for different nucleic acid-guided nucleases vary; however, PAMs typically are 2-10 or so base-pair sequences adjacent or in proximity to the target sequence and, depending on the nuclease, can be 5′ or 3′ to the target sequence. Engineering of the PAM-interacting domain of a nucleic acid-guided nuclease may allow for alteration of PAM specificity, improve target site recognition fidelity, decrease target site recognition fidelity, or increase the versatility of a nucleic acid-guided nuclease.

In most embodiments, genome editing of a cellular target sequence both introduces a desired DNA change to a cellular target sequence (an “intended” edit), e.g., the genomic DNA of a cell, and removes, mutates, or renders inactive a proto-spacer mutation (PAM) region in the cellular target sequence (an “immunizing edit”) thereby rendering the target site immune to further nuclease binding. Rendering the PAM at the cellular target sequence inactive precludes additional editing of the cell genome at that cellular target sequence, e.g., upon subsequent exposure to a nucleic acid-guided nuclease complexed with a synthetic guide nucleic acid in later rounds of editing. Thus, cells having the desired cellular target sequence edit and an altered PAM can be selected for by using a nucleic acid-guided nuclease complexed with a synthetic guide nucleic acid complementary to the cellular target sequence. Cells that did not undergo the first editing event will be cut rendering a double-stranded DNA break, and thus will not continue to be viable. The cells containing the desired cellular target sequence edit and PAM alteration will not be cut, as these edited cells no longer contain the necessary PAM site and will continue to grow and propagate.

As for the nuclease component of the nucleic acid-guided nuclease editing system described herein, a polynucleotide sequence encoding the nucleic acid-guided nuclease can be codon optimized for expression in particular cell types, such as bacterial, yeast, and mammalian cells. The choice of the nucleic acid-guided nuclease to be employed depends on many factors, such as what type of edit is to be made in the target sequence and whether an appropriate PAM is located close to the desired target sequence. CRISPR nucleases of use in the methods described herein include but are not limited to Cas9, Cas12/Cpf1, MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, MAD20, MAD2001, MAD2007, MAD2008, MAD2009, MAD2011, MAD2017, MAD2019, MAD297, MAD298, MAD299, or other MADzymes, variants thereof, and MADzyme systems (see U.S. Pat. Nos. 9,982,279; 10,337,028; 10,435,714; 10,011,849; 10,626,416; 10,604,746; 10,665,114; 10,640,754; 10,876,102; 10,883,077; 10,704,033; 10,745,678; 10,724,021; 10,767,169; and 10,870,761 for sequences and other details related to engineered and naturally-occurring MADzymes).

Another component of the nucleic acid-guided nuclease system is the repair template comprising homology to the cellular target sequence. For the present methods and compositions, the repair template typically is on the same vector and, in certain embodiments, in the same editing cassette, as the guide nucleic acid and may be under the control of the same promoter as the editing gRNA (that is, a single promoter driving the transcription of both the editing gRNA and the repair template). The repair template is designed to serve as a template for homologous recombination with a cellular target sequence nicked or cleaved by the nucleic acid-guided nuclease as a part of the gRNA/nuclease complex. A repair template polynucleotide may be of any suitable length, such as about or more than about 20, 25, 50, 75, 100, or more nucleotides in length. In certain preferred aspects, the repair template can be provided as an oligonucleotide of between 20-100 nucleotides, such as between 30-75 nucleotides. When optimally aligned, the repair template overlaps with (is complementary to) the cellular target sequence by, e.g., about 20, 25, 30, 35, 40, 50, 60, 70, 80, 90 or more nucleotides.

The repair template generally comprises two regions that are complementary to a portion of the cellular target sequence (e.g., homology arms). In certain embodiments of the present methods and compositions, the two homology arms flank an intended edit, e.g., at least one alteration as compared to the cellular target sequence, such as a large donor DNA sequence insertion, which may be part of the repair template. In certain embodiments, the repair template comprises two homology arms that do no flank the intended edit. In such embodiments, the homology arms may be encoded on an editing plasmid backbone, or in an editing cassette with the edit.

As described in relation to the gRNA, the repair template may be provided as part of a rationally-designed editing cassette, which is inserted into, e.g., an editing plasmid backbone, where the editing plasmid backbone may comprise a promoter to drive transcription of the editing gRNA and the repair template when the editing cassette is inserted into the plasmid backbone. In certain embodiments, there may be a single rationally-designed editing cassette, with a single editing gRNA/repair template pair, inserted into an editing plasmid and targeting a single region of the genome. In certain embodiments, there may be more than one, e.g., two, three, four, or more rationally-designed editing cassettes targeting the same or different regions of the genome; alternatively, a single rationally-designed editing cassette may comprise two to several editing gRNA/repair template pairs targeting the same or different regions of the genome, where each editing gRNA is under the control of separate different promoters, separate like promoters, or where all gRNAs/repair template pairs are under the control of a single promoter. In some embodiments the promoter driving transcription of the editing gRNA and the repair template (or driving more than one editing gRNA/repair template pair) is optionally an inducible promoter. In some embodiments, the promoter is a constitutive promoter.

An editing cassette may further comprise one or more primer binding sites. The primer binding sites are used to amplify the editing cassette by using oligonucleotide primers as described infra and may be biotinylated or otherwise labeled. In the current embodiments, the editing cassettes are a library of editing cassettes for, e.g., inserting large donor DNA sequences into different target locations in the population of cells via plasmid integration. Other embodiments envision performing successive rounds of editing where plasmids having different donor DNA sequences are embedded throughout the genome of a population of cells; that is, in round 1, an editing plasmid having donor DNA sequence 1 is embedded, in round 2, an editing plasmid having donor DNA sequence 2 is embedded, and so on. In addition, the library of editing cassettes may be cloned into plasmid backbones where, e.g., each different editing cassette or donor DNA sequence may be associated with a different barcode.

In certain embodiments, the plasmid and/or vector encoding components of the nucleic acid-guided nuclease system, e.g., the editing plasmid, further encodes a nucleic acid-guided nuclease comprising one or more nuclear localization sequences (NLSs), such as about or more than about 1, 2, 3, 4, 5, 6, 7, 8, 9, 10, or more NLSs, particularly as an element of the nuclease sequence. In some embodiments, the engineered nuclease comprises NLSs at or near the amino-terminus, NLSs at or near the carboxy-terminus, or a combination.

In certain embodiments, the editing plasmid or editing cassette further comprises a “landing pad” sequence, or a sequence of nucleotides comprising an enzyme recognition sequence, such as a recombinase, integrase, nuclease, or meganuclease recognition sequence. The landing pad can be leveraged to insert additional donor nucleic acid sequences, including additional plasmids, in subsequent and recursive editing operations.

In certain embodiments, the plasmids may further comprise one or more selectable markers to enable artificial selection of cells undergoing plasmid integration events. For example, in certain embodiments, the editing plasmids encode for one or more antibiotic resistance genes, such as ampicillin/carbenicillin and chloramphenicol resistance genes, thereby facilitating enrichment for cells undergoing plasmid integration events via depletion of the cell population. In other examples, plasmids may include an integrated GFP gene to enable phenotypic detection of plasmid integration events by flow cytometry, fluorescent cell imaging, etc.

### Nuclease-Directed Plasmid Integration Methods

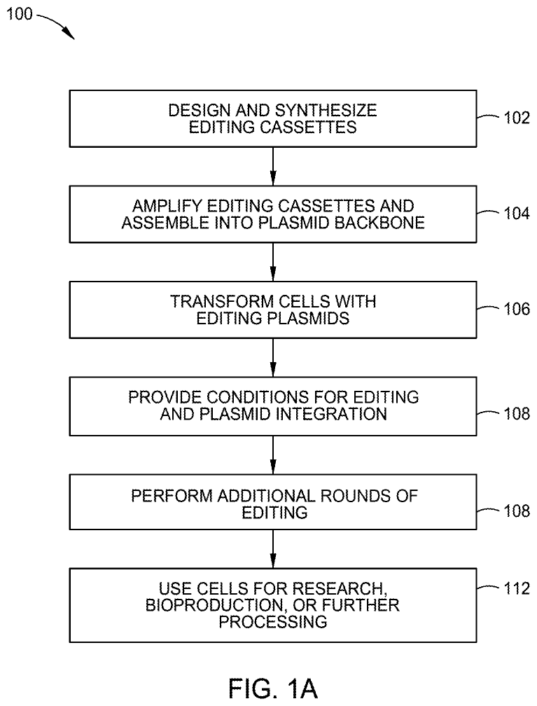

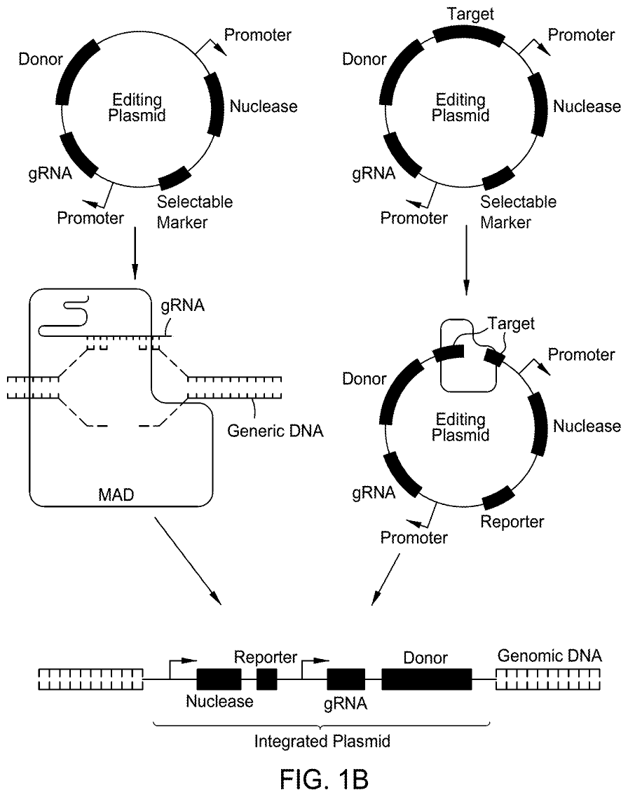

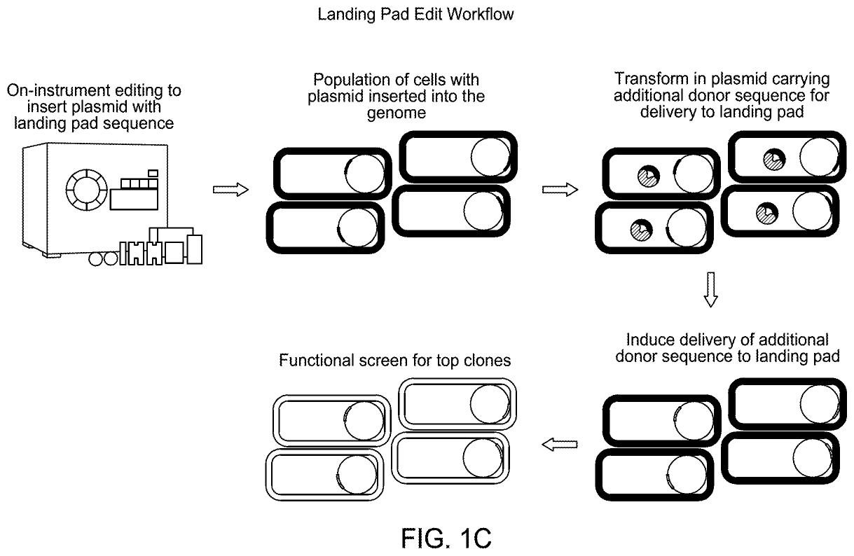

FIG. 1A is a simple process diagram of an exemplary method 100 for performing nucleic acid-guided nuclease editing in a population of cells to embed plasmids comprising donor nucleic acid sequences into target genetic loci in a population of cells. The method 100 may be utilized to insert large DNA payloads (e.g., >100 bp) into the target loci, including large DNA payloads comprising one or more genes. Looking at FIG. 1A, the method 100 begins by designing and synthesizing editing cassettes 102, which may each comprise a gRNA sequence, a donor nucleic acid sequence, and/or a repair template. In certain embodiments, the editing cassettes include other desired sequences, such as a barcode, primer amplification sites, and the like.

Once the individual editing cassettes have been synthesized, the individual cassettes are amplified (e.g., using primer amplification sites in the editing cassettes), purified, and assembled into plasmid backbones 104 to produce a library of editing plasmids. In certain embodiments, each plasmid backbone comprises a nucleic acid-guided nuclease coding sequence, as well as an optional selectable marker sequence and an optional barcode sequence. In certain other embodiments, the nucleic acid-guided nuclease coding sequence is included in the editing cassettes instead of the plasmid backbones, which are then assembled into the plasmid backbones. Alternatively, the coding sequence for the nucleic acid-guided nuclease may be located on another vector, such as an engine vector, that may be transformed into the cells before, at the same time as, or after the editing plasmids are transformed into the cells. Vectors chosen for the methods herein may vary depending on the type of cells being edited and analyzed, where the vectors include, e.g., plasmids, BACs, YACs, viral vectors and synthetic chromosomes. In yet other alternatives, the cells may be transformed with a single combined engine/editing plasmid comprising all components required to perform genome editing to integrate the plasmids, or the cells may already be expressing the nuclease (e.g., the coding sequence for the nuclease may be stably integrated into the cellular genome). In still other embodiments, the nuclease may be delivered to the cell as a protein.

The cells of interest useful in the methods herein are any cells, including bacterial, yeast and animal (including mammalian) cells. Before being transformed by the editing plasmids and other vectors, the cells are often grown in culture for several passages. Cell culture is the process by which cells are grown under controlled conditions, almost always outside the cell's natural environment. For bacterial and yeast cells, the cells are typically grown in a defined medium in bulk culture. For mammalian cells, culture conditions typically vary somewhat for each cell type but generally include a medium and additives that supply essential nutrients such as amino acids, carbohydrates, vitamins, minerals, growth factors, hormones, and gases such as, e.g., 02 and CO2. In addition to providing nutrients, the medium typically regulates the physio-chemical environment via a pH buffer and most cells are grown at 37° C. Many mammalian cells require or prefer a surface or artificial substrate on which to grow (e.g., adherent cells), whereas other cells such as hematopoietic cells and some adherent cells can be grown in or adapted to grow in suspension. Adherent cells often are grown in 2D monolayer cultures in petri dishes or flasks, but some adherent cells can grow in suspension cultures to higher density than would be possible in 2D cultures. “Passages” generally refers to transferring a small number of cells to a fresh substrate with fresh medium, or, in the case of suspension cultures, transferring a small volume of the culture to a larger volume of medium.

The cells of choice are provided and are transformed with the library of editing plasmids 106 (and in certain embodiments, other vectors), thereby creating a library of transformed cells. The library of editing plasmids comprises plasmid backbones each “carrying” one or more editing cassettes. For single edits where one donor nucleic acid sequence is inserted per cell, the edit is the same for every cassette in the library, but the edits may be targeted to different locations around the genome. The library of editing cassettes may have tens, hundreds, thousands, tens of thousands or more different editing cassettes (in this case, tens, hundreds, thousands, tens of thousands or more different guides), where the donor nucleic acid sequences are the same but the gRNA and/or homology arms are different for insertion into different genomic target loci.

As used herein, transformation is intended to generically include a variety of art-recognized techniques for introducing an exogenous nucleic acid sequence (e.g., an engine vector and/or editing plasmid) into a target cell, and the term “transformation” as used herein includes all transformation, transduction, and transfection techniques. Such methods include, but are not limited to, electroporation, lipofection, optoporation, injection, microprecipitation, microinjection, liposomes, particle bombardment, sonoporation, laser-induced poration, bead transfection, calcium phosphate or calcium chloride co-precipitation, or DEAE-dextran-mediated transfection. Cells can also be prepared for vector uptake using, e.g., a sucrose, sorbitol or glycerol wash. Additionally, hybrid techniques that exploit the capabilities of mechanical and chemical transfection methods can be used, e.g., magnetofection, a transfection methodology that combines chemical transfection with mechanical methods. In another example, cationic lipids may be deployed in combination with gene guns or electroporators. Suitable materials and methods for transforming or transfecting target cells can be found, e.g., in Green and Sambrook, Molecular Cloning: A Laboratory Manual, 4th ed., Cold Spring Harbor Laboratory Press, Cold Spring Harbor, N.Y. (2014).

Once transformed 106, the cells are allowed to recover and selection is optionally performed to select for cells transformed with the editing vector, which most often comprises a selectable marker. Selectable markers and selection medium are employed to select for cells that have received the vector backbone. Commonly used selectable markers include drug selectable markers such as ampicillin/carbenicillin, kanamycin, chloramphenicol, erythromycin, tetracycline, gentamicin, bleomycin, streptomycin, puromycin, hygromycin, blasticidin, and GF18.

At a next step 108, conditions are provided to facilitate editing, and thus, integration of editing plasmids into the cell genome. “Providing conditions” includes incubation of the cells in appropriate medium and may also include providing conditions to facilitate, or even induce via an inducible promoter, transcription of the gRNA and nucleic acid-guided nuclease. For example, if one or more components of the editing machinery (e.g., editing cassette and nuclease) are under the control of inducible promoters, conditions are provided to induce editing. If none of the components of the editing machinery are under the control of an inducible promoter, editing may proceed immediately after transformation. During the editing process, many cells may die due to double-strand breaks in the genome that are a consequence of the editing process. Of the cells that do survive editing and continue to grow, the surviving cells will comprise an integrated editing plasmid, which itself may comprise a large DNA payload.

In certain embodiments, additional rounds of recursive editing are performed 110 after the initial editing takes place. For example, the cells may be grown (for 1-4 hours, or typically 8, 10 or 14 hours in rich medium and optional antibiotic selection at 15-37° C. depending on cell type) and prepared for another round of transformation, this time with a plasmid or vector carrying a different, additional donor DNA sequence for insertion at, e.g., a landing pad integrated in the cellular genome with the first plasmid, or at a different target locus in the cellular genome. Accordingly, steps 102-108 may be repeated as needed. The utilization of landing pads for recursive editing is described in more detail below with regards to FIG. 1C. Once all desired editing is complete, the cells are allowed to recover and may then be, e.g., utilized in research, utilized for bioproduction systems, exposed to further processing, etc.

FIG. 1B is a simplified exemplary depiction of nuclease-directed genome editing wherein an editing plasmid carrying one or more donor nucleic acid sequences is integrated into a cell genome. In particular, two different exemplary mechanisms are shown. In a first example (left), an editing plasmid comprises coding sequences for a nuclease (under the control of a promoter), a selection marker, and a gRNA designed to bind to a target locus of the cell genome (also under the control of a promoter), as well as a donor nucleic acid sequence. Although not shown, the editing plasmid further comprises regions of homology to the target locus, e.g., HDR sequences or homology arms. In this example, during editing, the coded nuclease (e.g., MAD7 or similar MADzyme) associates with the coded gRNA to form a recognition complex that specifically binds to and generates a double-stranded break in the genomic DNA at a target locus. Thereafter, the regions of homology on the editing plasmid facilitate integration of the plasmid into the target locus via homology-directed repair or other recombination pathways.

In the example on the right, the editing plasmid comprises similar components to the plasmid described, including coding sequences for a nuclease (under the control of a promoter), a selection marker, and a gRNA (also under the control of a promoter), as well as a donor nucleic acid sequence and regions of homology to a target locus in the cell genome. However, the editing plasmid on the right further includes a self-targeting sequence, to which the embedded gRNA or other transformed gRNA is designed to bind. Accordingly, during editing, the editing plasmid is cut and linearized, upon which the regions of homology on the editing plasmid facilitate integration of the plasmid into the target locus via homology-directed repair or other recombination pathways.

FIG. 1C is a simplified diagram of a process for utilizing and leveraging landing pads to insert donor DNA sequences, and in certain embodiments, additional plasmids, in additional rounds of editing, as briefly described above in FIG. 1A. The method begins with editing of a cell population to integrate at least a first plasmid having a landing pad sequence into the cellular genome (e.g., utilizing Method 100). In certain embodiments, editing is carried out using a library of editing plasmids further comprising CREATE editing cassettes, preferably in an automated manner using an instrument (depicted at left in FIG. 1C) such as described U.S. Pat. No. 10,253,316; 10,329,559; 10,323,242; 10,421,959; 10,465,185; 10,519,437; 10,584,333; 10,584,334; 10,647,982; 10,689,645; 10,738,301; 10,738,663; 10,947,532; 10,894,958; 10,954,512; and 11,034,953; and U.S. Ser. No. 17/239,540. After editing, the population of cells comprise a genome with an integrated plasmid having a landing pad, which may in certain embodiments be integrated into different loci around the genome, depicted as a black bar on a circular genome in the cell.

Following insertion of the plasmid having a landing pad the genome, the cells are then transformed with a plasmid or other vector carrying an additional donor DNA sequence to be delivered to the landing pad (depicted as striped bars on the vectors in the cells). As described above, transformation is intended to generically include a variety of art-recognized techniques for introducing an exogenous nucleic acid sequence (e.g., an engine and/or editing vector) into a target cell, and the term “transformation” as used herein includes all transformation, transduction, and transfection techniques. Each plasmid or vector may comprise 1) a coding sequence for an appropriate recombinase/integrase or meganuclease targeting the landing pad recognition sequence; and 2) either a large donor DNA sequence flanked by either the recombinase or integrase recognition sequence for recombinase/integrase-mediated insertion into the landing pad in the genome, or a large donor DNA sequence flanked by homology arm sequences for HDR-mediated insertion into the genome via the meganuclease. In an optional (but preferred) step, the plasmid or vector also comprises a coding sequence for a selection marker and the cells are selected after transformation.

After transformation and optional selection, delivery of the large donor DNA sequences to the landing pads in the cells is induced by inducing expression of the recombinase/integrase or meganuclease. The cells with the DNA payload delivered to the landing pads are allowed to recover and grow and then are screened. Note that after delivery of the donor DNA sequence to the landing pads, the black bar on the chromosome in the cells is transformed into a striped bar. Screening for proper integration of the donor DNA sequences includes but is not limited to 1) polymerase chain reaction (PCR) analysis with appropriate primer sets used to assess whether the delivery vector was correctly integrated at the target site; 2) assessment of activity of the nucleic acid of interest, including but not limited to a metabolic test, measurement of transcript level, a phenotypic assay, or detection of a protein product using an antibody specific to the protein product; and/or 3) DNA sequencing of the integrated sequence. Exemplary applications of the present compositions and methods include genome-wide delivery of large-insert promoter libraries; delivery of heterologous genes or pathways to a large number of genomic locations enabling examination of location-dependent expression effects; and delivery of fusion-protein partners to multiple loci around the genome.

### Automated Cell Processing Instruments and Modules to Perform Nucleic Acid-Guided Nuclease or Nickase Editing in Cells to Integrate Plasmids

### Automated Cell Processing Instruments

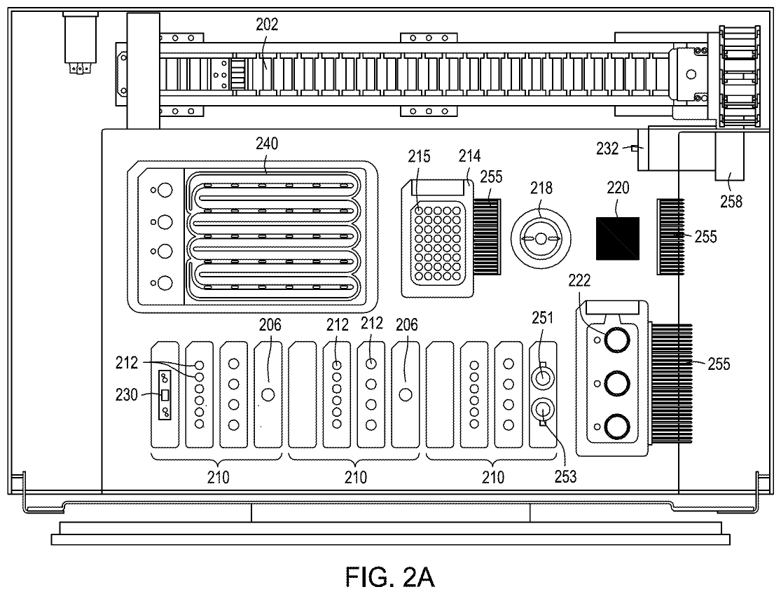

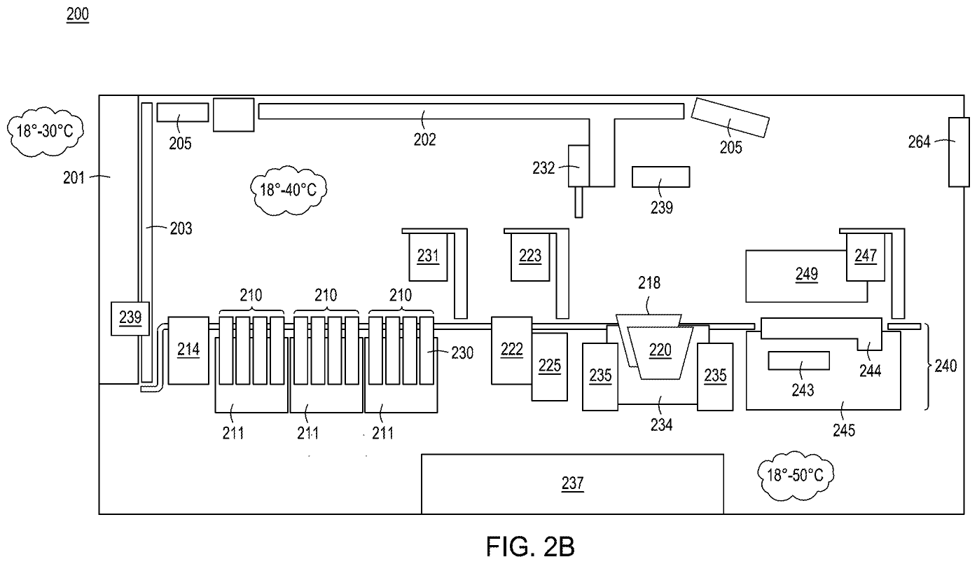

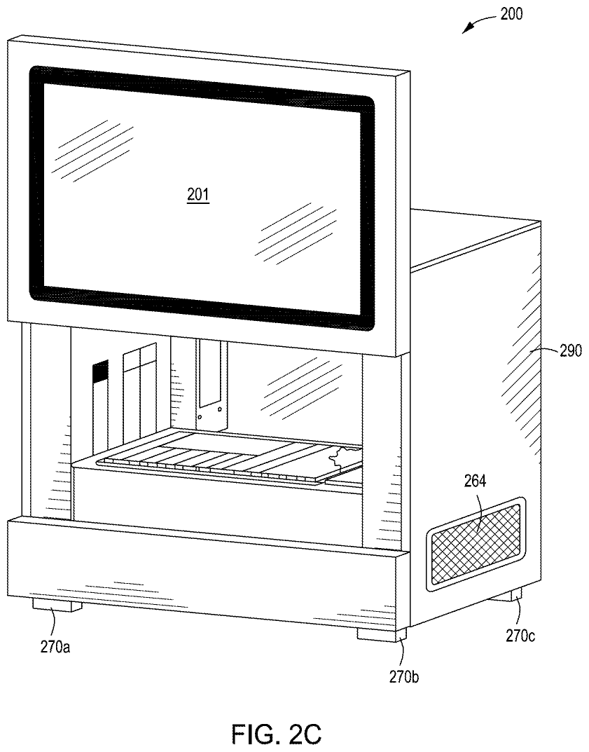

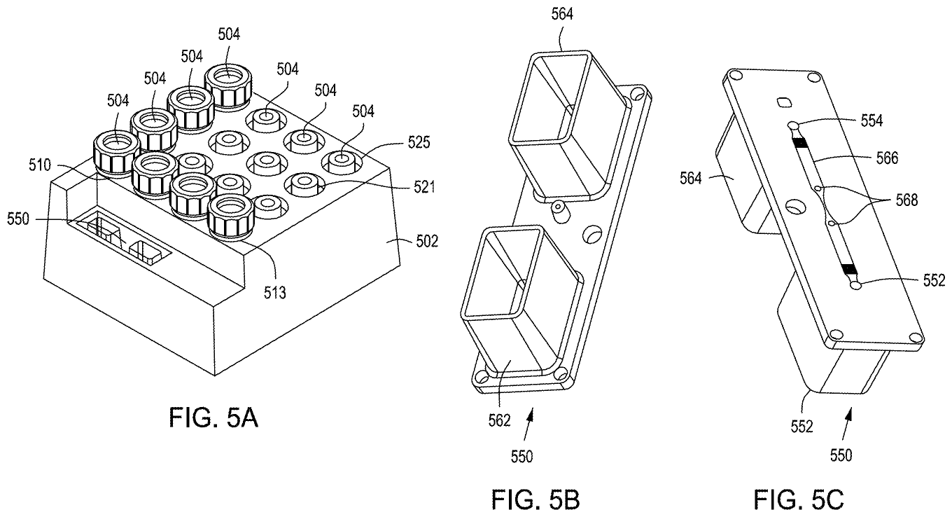

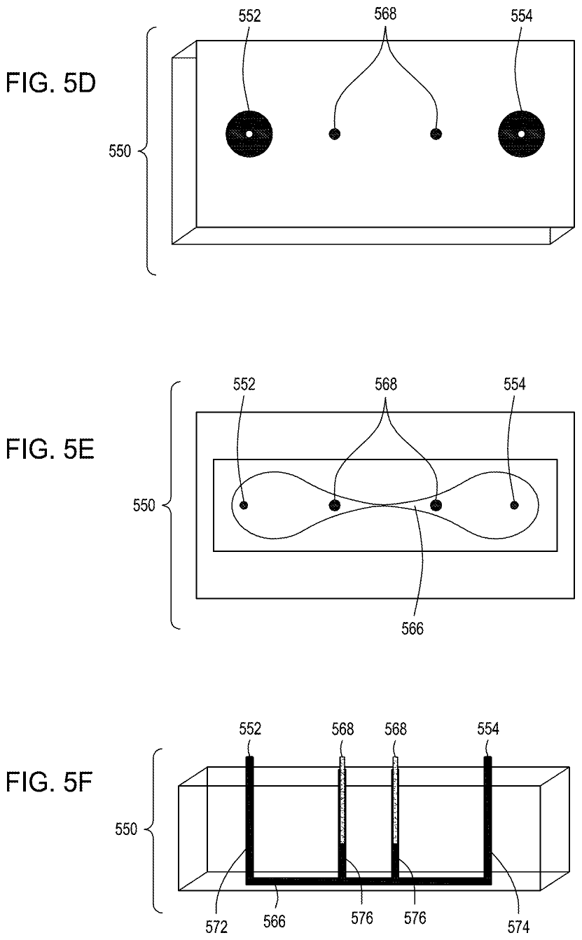

FIG. 2A depicts an exemplary automated multi-module cell processing instrument 200 to, e.g., perform one of the exemplary workflows described herein. The instrument 200, for example, may be and preferably is designed as a stand-alone desktop instrument for use within a laboratory environment. The instrument 200 may incorporate a mixture of reusable and disposable components for performing the various integrated processes in conducting automated genome cleavage and/or editing in cells without human intervention. Illustrated is a gantry 202, providing an automated mechanical motion system (actuator) (not shown) that supplies XYZ axis motion control to, e.g., an automated (i.e., robotic) liquid handling system 258 including, e.g., an air displacement pipettor 232 which allows for cell processing among multiple modules without human intervention. In some automated multi-module cell processing instruments, the air displacement pipettor 232 is moved by gantry 202 and the various modules and reagent cartridges remain stationary; however, in other embodiments, the liquid handling system 258 may stay stationary while the various modules and reagent cartridges are moved. Also included in the automated multi-module cell processing instrument 200 are reagent cartridges 210 comprising reservoirs 212 and transformation module 230 (e.g., a flow-through electroporation device as described in detail in relation to FIGS. 5B-5F), as well as wash reservoirs 206, cell input reservoir 251 and cell output reservoir 253. The wash reservoirs 206 may be configured to accommodate large tubes, for example, wash solutions, or solutions that are used often throughout an iterative process. Although two of the reagent cartridges 210 comprise a wash reservoir 206 in FIG. 2A, the wash reservoirs instead could be included in a wash cartridge where the reagent and wash cartridges are separate cartridges. In such a case, the reagent cartridge 210 and wash cartridge 204 may be identical except for the consumables (reagents or other components contained within the various inserts) inserted therein.

In some implementations, the reagent cartridges 210 are disposable kits comprising reagents and cells for use in the automated multi-module cell processing instrument 200. For example, a user may open and position each of the reagent cartridges 210 comprising various desired inserts and reagents within the chassis of the automated multi-module cell processing instrument 200 prior to activating cell processing. Further, each of the reagent cartridges 210 may be inserted into receptacles in the chassis having different temperature zones appropriate for the reagents contained therein.

Also illustrated in FIG. 2A is the robotic liquid handling system 258 including the gantry 202 and air displacement pipettor 232. In some examples, the robotic handling system 258 may include an automated liquid handling system such as those manufactured by Tecan Group Ltd. of Mannedorf, Switzerland, Hamilton Company of Reno, Nev. (see, e.g., WO2018015544A1), or Beckman Coulter, Inc. of Fort Collins, Colo. (see, e.g., US20160018427A1). Pipette tips may be provided in a pipette transfer tip supply (not shown) for use with the air displacement pipettor 232.

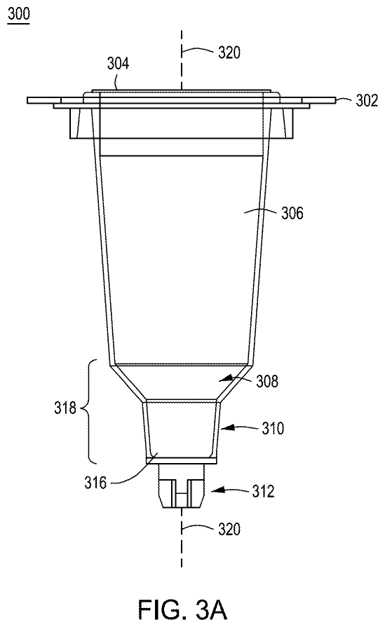

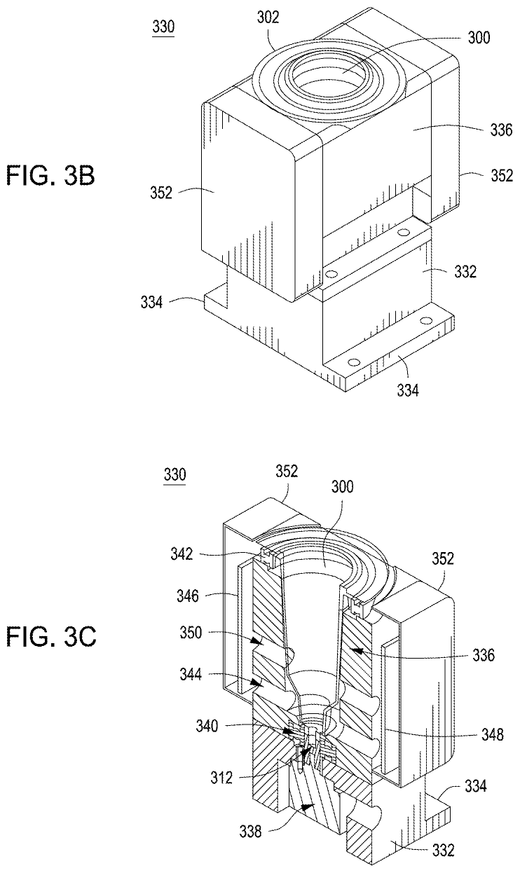

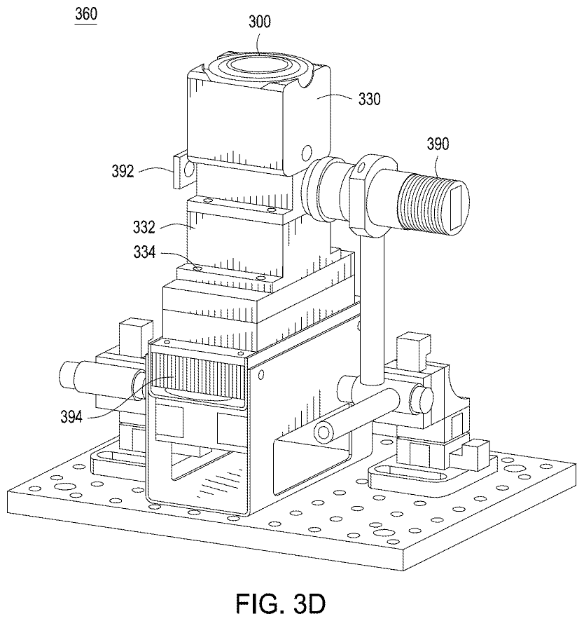

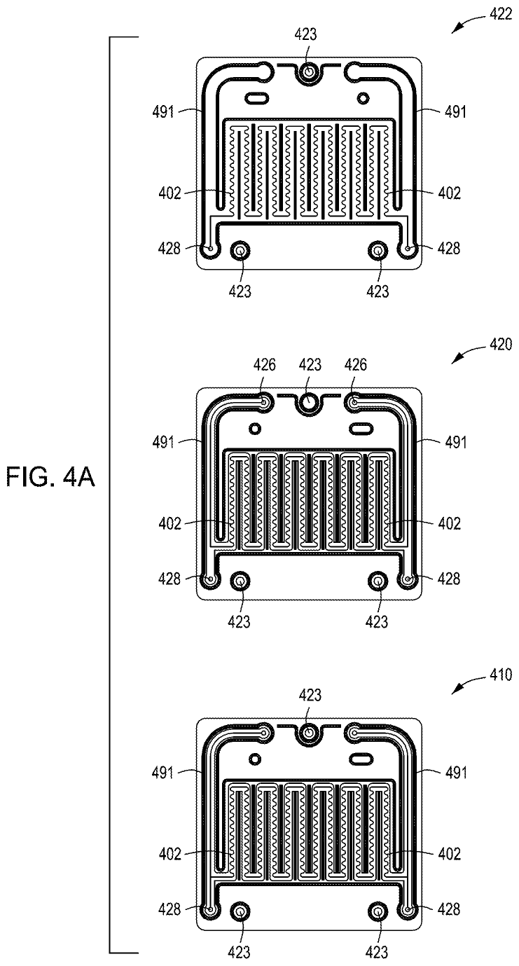

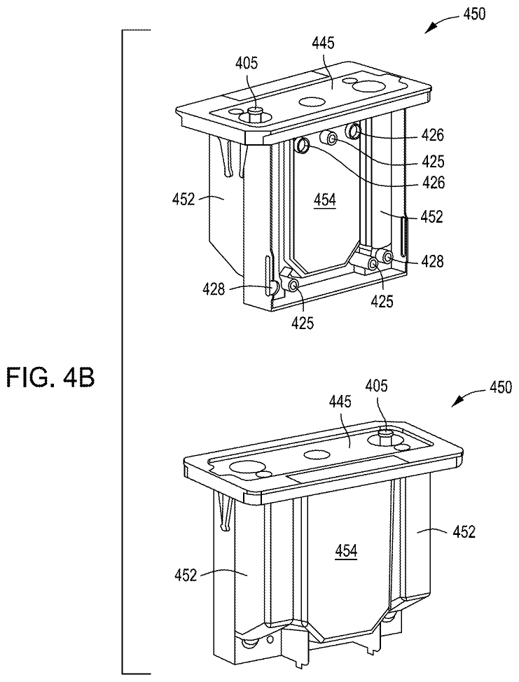

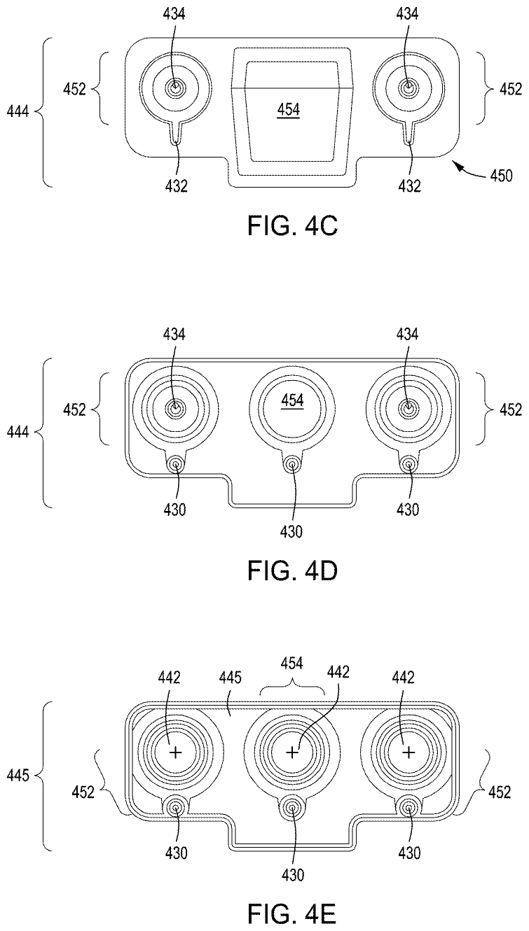

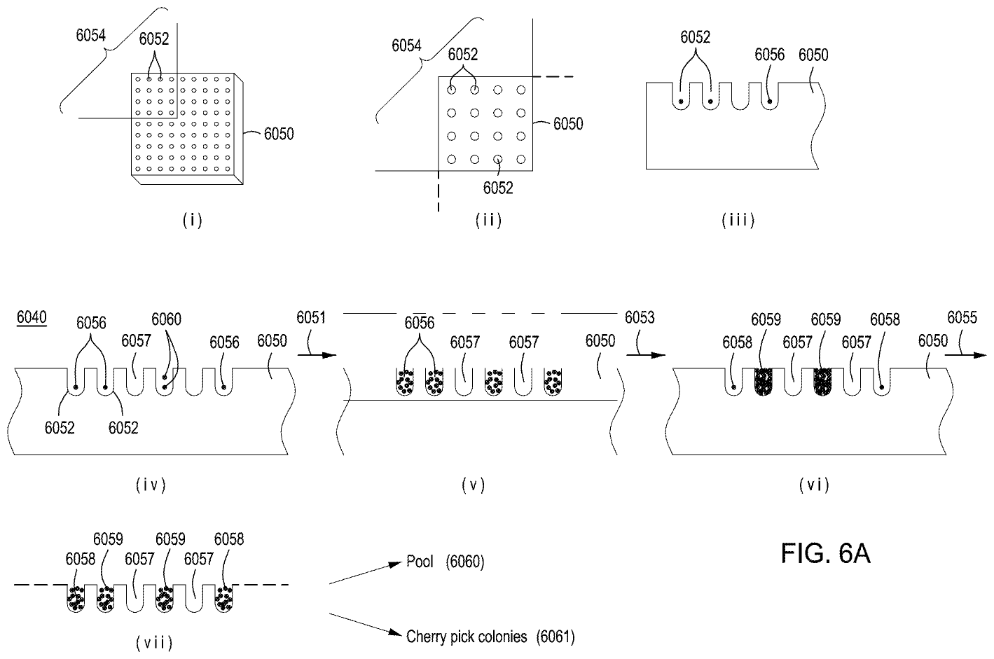

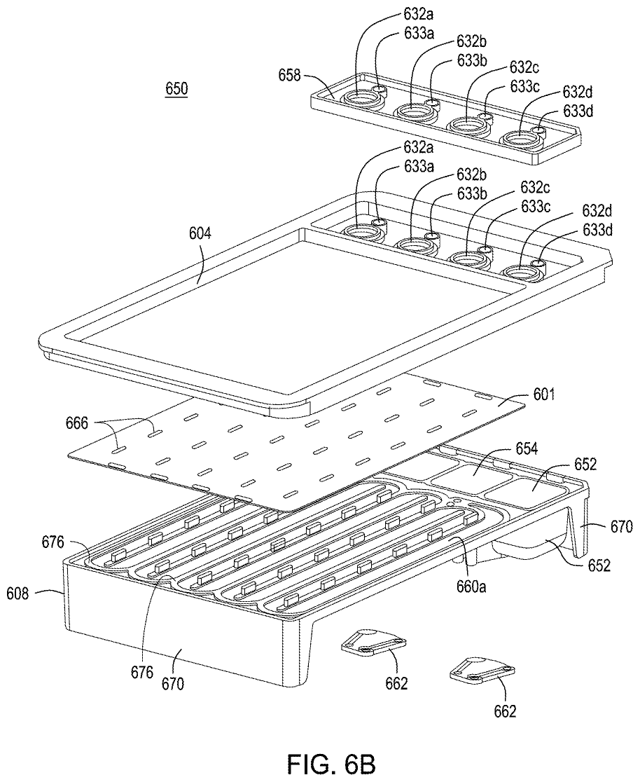

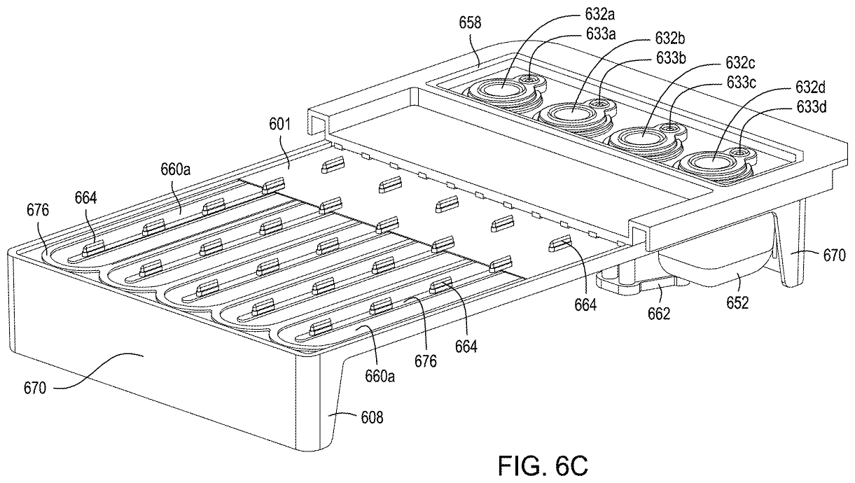

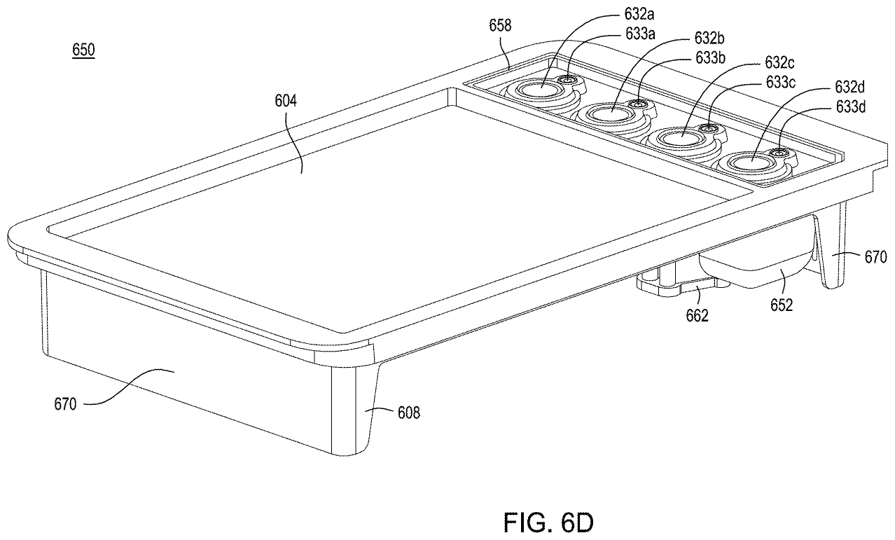

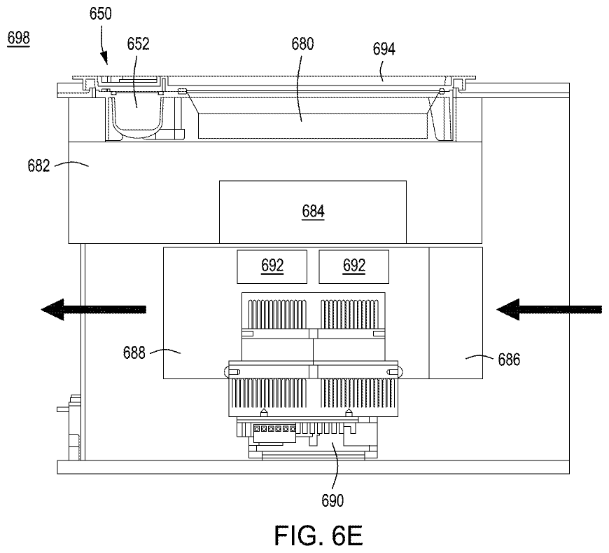

Inserts or components of the reagent cartridges 210, in some implementations, are marked with machine-readable indicia (not shown), such as bar codes, for recognition by the robotic handling system 258. For example, the robotic liquid handling system 258 may scan one or more inserts within each of the reagent cartridges 210 to confirm contents. In other implementations, machine-readable indicia may be marked upon each reagent cartridge 210, and a processing system (not shown, but see element 237 of FIG. 2B) of the automated multi-module cell processing instrument 200 may identify a stored materials map based upon the machine-readable indicia. In the embodiment illustrated in FIG. 2A, a cell growth module comprises a cell growth vial 218 (described in greater detail below in relation to FIGS. 3A-3D). Additionally seen is the TFF module 222 (described above in detail in relation to FIGS. 4A-4E). Also illustrated as part of the automated multi-module cell processing instrument 200 of FIG. 2A is a singulation module 240 (e.g., a solid wall isolation, incubation and normalization device (SWIIN device) is shown here) described herein in relation to FIGS. 6C-6E, served by, e.g., robotic liquid handing system 258 and air displacement pipettor 232. Additionally seen is a selection module 220. Also note the placement of three heatsinks 255.

FIG. 2B is a simplified representation of the contents of the exemplary multi-module cell processing instrument 200 depicted in FIG. 2A. Cartridge-based source materials (such as in reagent cartridges 210), for example, may be positioned in designated areas on a deck of the instrument 200 for access by an air displacement pipettor 232. The deck of the multi-module cell processing instrument 200 may include a protection sink such that contaminants spilling, dripping, or overflowing from any of the modules of the instrument 200 are contained within a lip of the protection sink. Also seen are reagent cartridges 210, which are shown disposed with thermal assemblies 211 which can create temperature zones appropriate for different regions. Note that one of the reagent cartridges also comprises a flow-through electroporation device 230 (FTEP), served by FTEP interface (e.g., manifold arm) and actuator 231. Also seen is TFF module 222 with adjacent thermal assembly 225, where the TFF module is served by TFF interface (e.g., manifold arm) and actuator 233. Thermal assemblies 225, 235, and 245 encompass thermal electric devices such as Peltier devices, as well as heatsinks, fans and coolers. The rotating growth vial 218 is within a growth module 234, where the growth module is served by two thermal assemblies 235. Selection module is seen at 220. Also seen is the SWIIN module 240, comprising a SWIIN cartridge 241, where the SWIIN module also comprises a thermal assembly 245, illumination 243 (in this embodiment, backlighting), evaporation and condensation control 249, and where the SWIIN module is served by SWIIN interface (e.g., manifold arm) and actuator 247. Also seen in this view is touch screen display 201, display actuator 203, illumination 205 (one on either side of multi-module cell processing instrument 200), and cameras 239 (one illumination device on either side of multi-module cell processing instrument 200). Finally, element 237 comprises electronics, such as circuit control boards, high-voltage amplifiers, power supplies, and power entry; as well as pneumatics, such as pumps, valves and sensors.

FIG. 2C illustrates a front perspective view of multi-module cell processing instrument 200 for use in as a desktop version of the automated multi-module cell processing instrument 200. For example, a chassis 290 may have a width of about 24-48 inches, a height of about 24-48 inches and a depth of about 24-48 inches. Chassis 290 may be and preferably is designed to hold all modules and disposable supplies used in automated cell processing and to perform all processes required without human intervention; that is, chassis 290 is configured to provide an integrated, stand-alone automated multi-module cell processing instrument. As illustrated in FIG. 2C, chassis 290 includes touch screen display 201, cooling grate 264, which allows for air flow via an internal fan (not shown). The touch screen display provides information to a user regarding the processing status of the automated multi-module cell processing instrument 200 and accepts inputs from the user for conducting the cell processing. In this embodiment, the chassis 290 is lifted by adjustable feet 270a, 270b, 270c and 270d (feet 270a-270c are shown in this FIG. 2C). Adjustable feet 270a-270d, for example, allow for additional air flow beneath the chassis 290.

Inside the chassis 290, in some implementations, will be most or all of the components described in relation to FIGS. 2A and 2B, including the robotic liquid handling system disposed along a gantry, reagent cartridges 210 including a flow-through electroporation device, a rotating growth vial 218 in a cell growth module 234, a tangential flow filtration module 222, a SWIIN module 240 as well as interfaces and actuators for the various modules. In addition, chassis 290 houses control circuitry, liquid handling tubes, air pump controls, valves, sensors, thermal assemblies (e.g., heating and cooling units) and other control mechanisms. For examples of multi-module cell processing instruments, see U.S. Pat. No. 10,253,316; 10,329,559; 10,323,242; 10,421,959; 10,465,185; 10,519,437; 10,584,333; 10,584,334; 10,647,982; 10,689,645; 10,738,301; 10,738,663 and U.S. Ser. Nos. 16/412,175 and 16/988,694, all of which are herein incorporated by reference in their entirety.

### The Rotating Cell Growth Module

FIG. 3A shows one embodiment of a rotating growth vial 300 for use with the cell growth device and in the automated multi-module cell processing instruments described herein. The rotating growth vial 300 is an optically-transparent container having an open end 304 for receiving liquid media and cells, a central vial region 306 that defines the primary container for growing cells, a tapered-to-constricted region 318 defining at least one light path 310, a closed end 316, and a drive engagement mechanism 312. The rotating growth vial 300 has a central longitudinal axis 320 around which the vial rotates, and the light path 310 is generally perpendicular to the longitudinal axis of the vial. The first light path 310 is positioned in the lower constricted portion of the tapered-to-constricted region 318. Optionally, some embodiments of the rotating growth vial 300 have a second light path 308 in the tapered region of the tapered-to-constricted region 318. Both light paths in this embodiment are positioned in a region of the rotating growth vial that is constantly filled with the cell culture (cells+growth media) and are not affected by the rotational speed of the growth vial. The first light path 310 is shorter than the second light path 308 allowing for sensitive measurement of OD values when the OD values of the cell culture in the vial are at a high level (e.g., later in the cell growth process), whereas the second light path 308 allows for sensitive measurement of OD values when the OD values of the cell culture in the vial are at a lower level (e.g., earlier in the cell growth process).

The drive engagement mechanism 312 engages with a motor (not shown) to rotate the vial. In some embodiments, the motor drives the drive engagement mechanism 312 such that the rotating growth vial 300 is rotated in one direction only, and in other embodiments, the rotating growth vial 300 is rotated in a first direction for a first amount of time or periodicity, rotated in a second direction (i.e., the opposite direction) for a second amount of time or periodicity, and this process may be repeated so that the rotating growth vial 300 (and the cell culture contents) are subjected to an oscillating motion. Further, the choice of whether the culture is subjected to oscillation and the periodicity therefor may be selected by the user. The first amount of time and the second amount of time may be the same or may be different. The amount of time may be 1, 2, 3, 4, 5, or more seconds, or may be 1, 2, 3, 4 or more minutes. In another embodiment, in an early stage of cell growth the rotating growth vial 400 may be oscillated at a first periodicity (e.g., every 60 seconds), and then a later stage of cell growth the rotating growth vial 300 may be oscillated at a second periodicity (e.g., every one second) different from the first periodicity.

The rotating growth vial 300 may be reusable or, preferably, the rotating growth vial is consumable. In some embodiments, the rotating growth vial is consumable and is presented to the user pre-filled with growth medium, where the vial is hermetically sealed at the open end 304 with a foil seal. A medium-filled rotating growth vial packaged in such a manner may be part of a kit for use with a stand-alone cell growth device or with a cell growth module that is part of an automated multi-module cell processing system. To introduce cells into the vial, a user need only pipette up a desired volume of cells and use the pipette tip to punch through the foil seal of the vial. Open end 304 may optionally include an extended lip 302 to overlap and engage with the cell growth device. In automated systems, the rotating growth vial 300 may be tagged with a barcode or other identifying means that can be read by a scanner or camera (not shown) that is part of the automated system.

The volume of the rotating growth vial 300 and the volume of the cell culture (including growth medium) may vary greatly, but the volume of the rotating growth vial 300 must be large enough to generate a specified total number of cells. In practice, the volume of the rotating growth vial 300 may range from 1-250 mL, 2-100 mL, from 5-80 mL, 10-50 mL, or from 12-35 mL. Likewise, the volume of the cell culture (cells+growth media) should be appropriate to allow proper aeration and mixing in the rotating growth vial 400. Proper aeration promotes uniform cellular respiration within the growth media. Thus, the volume of the cell culture should be approximately 5-85% of the volume of the growth vial or from 20-60% of the volume of the growth vial. For example, for a 30 mL growth vial, the volume of the cell culture would be from about 1.5 mL to about 26 mL, or from 6 mL to about 18 mL.

The rotating growth vial 300 preferably is fabricated from a bio-compatible optically transparent material—or at least the portion of the vial comprising the light path(s) is transparent. Additionally, material from which the rotating growth vial is fabricated should be able to be cooled to about 4° C. or lower and heated to about 55° C. or higher to accommodate both temperature-based cell assays and long-term storage at low temperatures. Further, the material that is used to fabricate the vial must be able to withstand temperatures up to 55° C. without deformation while spinning. Suitable materials include cyclic olefin copolymer (COC), glass, polyvinyl chloride, polyethylene, polyamide, polypropylene, polycarbonate, poly(methyl methacrylate (PMMA), polysulfone, polyurethane, and co-polymers of these and other polymers. Preferred materials include polypropylene, polycarbonate, or polystyrene. In some embodiments, the rotating growth vial is inexpensively fabricated by, e.g., injection molding or extrusion.

FIG. 3B is a perspective view of one embodiment of a cell growth device 330. FIG. 3C depicts a cut-away view of the cell growth device 330 from FIG. 3B. In both figures, the rotating growth vial 300 is seen positioned inside a main housing 336 with the extended lip 302 of the rotating growth vial 300 extending above the main housing 336. Additionally, end housings 352, a lower housing 332 and flanges 334 are indicated in both figures. Flanges 334 are used to attach the cell growth device 330 to heating/cooling means or other structure (not shown). FIG. 3C depicts additional detail. In FIG. 3C, upper bearing 342 and lower bearing 340 are shown positioned within main housing 336. Upper bearing 342 and lower bearing 340 support the vertical load of rotating growth vial 300. Lower housing 332 contains the drive motor 338. The cell growth device 330 of FIG. 3C comprises two light paths: a primary light path 344, and a secondary light path 350. Light path 344 corresponds to light path 310 positioned in the constricted portion of the tapered-to-constricted portion of the rotating growth vial 300, and light path 350 corresponds to light path 308 in the tapered portion of the tapered-to-constricted portion of the rotating growth via 316. Light paths 310 and 308 are not shown in FIG. 3C but may be seen in FIG. 3A. In addition to light paths 344 and 340, there is an emission board 348 to illuminate the light path(s), and detector board 346 to detect the light after the light travels through the cell culture liquid in the rotating growth vial 300.

The motor 338 engages with drive mechanism 312 and is used to rotate the rotating growth vial 300. In some embodiments, motor 338 is a brushless DC type drive motor with built-in drive controls that can be set to hold a constant revolution per minute (RPM) between 0 and about 3000 RPM. Alternatively, other motor types such as a stepper, servo, brushed DC, and the like can be used. Optionally, the motor 338 may also have direction control to allow reversing of the rotational direction, and a tachometer to sense and report actual RPM. The motor is controlled by a processor (not shown) according to, e.g., standard protocols programmed into the processor and/or user input, and the motor may be configured to vary RPM to cause axial precession of the cell culture thereby enhancing mixing, e.g., to prevent cell aggregation, increase aeration, and optimize cellular respiration.

Main housing 336, end housings 352 and lower housing 332 of the cell growth device 330 may be fabricated from any suitable, robust material including aluminum, stainless steel, and other thermally conductive materials, including plastics. These structures or portions thereof can be created through various techniques, e.g., metal fabrication, injection molding, creation of structural layers that are fused, etc. Whereas the rotating growth vial 300 is envisioned in some embodiments to be reusable, but preferably is consumable, the other components of the cell growth device 330 are preferably reusable and function as a stand-alone benchtop device or as a module in a multi-module cell processing system.

The processor (not shown) of the cell growth device 330 may be programmed with information to be used as a “blank” or control for the growing cell culture. A “blank” or control is a vessel containing cell growth medium only, which yields 100% transmittance and 0 OD, while the cell sample will deflect light rays and will have a lower percent transmittance and higher OD. As the cells grow in the media and become denser, transmittance will decrease and OD will increase. The processor (not shown) of the cell growth device 330—may be programmed to use wavelength values for blanks commensurate with the growth media typically used in cell culture (whether, e.g., mammalian cells, bacterial cells, animal cells, yeast cells, etc.). Alternatively, a second spectrophotometer and vessel may be included in the cell growth device 330, where the second spectrophotometer is used to read a blank at designated intervals.

FIG. 3D illustrates a cell growth device 330 as part of an assembly comprising the cell growth device 330 of FIG. 3B coupled to light source 390, detector 392, and thermal components 394. The rotating growth vial 300 is inserted into the cell growth device. Components of the light source 390 and detector 392 (e.g., such as a photodiode with gain control to cover 5-log) are coupled to the main housing of the cell growth device. The lower housing 332 that houses the motor that rotates the rotating growth vial 300 is illustrated, as is one of the flanges 334 that secures the cell growth device 330 to the assembly. Also, the thermal components 394 illustrated are a Peltier device or thermoelectric cooler. In this embodiment, thermal control is accomplished by attachment and electrical integration of the cell growth device 330 to the thermal components 394 via the flange 334 on the base of the lower housing 332. Thermoelectric coolers are capable of “pumping” heat to either side of a junction, either cooling a surface or heating a surface depending on the direction of current flow. In one embodiment, a thermistor is used to measure the temperature of the main housing and then, through a standard electronic proportional-integral-derivative (PID) controller loop, the rotating growth vial 300 is controlled to approximately +/−0.5° C.

In use, cells are inoculated (cells can be pipetted, e.g., from an automated liquid handling system or by a user) into pre-filled growth media of a rotating growth vial 300 by piercing though the foil seal or film. The programmed software of the cell growth device 330 sets the control temperature for growth, typically 30° C., then slowly starts the rotation of the rotating growth vial 300. The cell/growth media mixture slowly moves vertically up the wall due to centrifugal force allowing the rotating growth vial 300 to expose a large surface area of the mixture to a normal oxygen environment. The growth monitoring system takes either continuous readings of the OD or OD measurements at pre-set or pre-programmed time intervals. These measurements are stored in internal memory and if requested the software plots the measurements versus time to display a growth curve. If enhanced mixing is required, e.g., to optimize growth conditions, the speed of the vial rotation can be varied to cause an axial precession of the liquid, and/or a complete directional change can be performed at programmed intervals. The growth monitoring can be programmed to automatically terminate the growth stage at a pre-determined OD, and then quickly cool the mixture to a lower temperature to inhibit further growth.

One application for the cell growth device 330 is to constantly measure the optical density of a growing cell culture. One advantage of the described cell growth device is that optical density can be measured continuously (kinetic monitoring) or at specific time intervals; e.g., every 5, 10, 15, 20, 30 45, or 60 seconds, or every 1, 2, 3, 4, 5, 6, 7, 8, 9, or 10 minutes. While the cell growth device 330 has been described in the context of measuring the optical density (OD) of a growing cell culture, it should, however, be understood by a skilled artisan given the teachings of the present specification that other cell growth parameters can be measured in addition to or instead of cell culture OD. As with optional measure of cell growth in relation to the solid wall device or module described supra, spectroscopy using visible, UV, or near infrared (NIR) light allows monitoring the concentration of nutrients and/or wastes in the cell culture and other spectroscopic measurements may be made; that is, other spectral properties can be measured via, e.g., dielectric impedance spectroscopy, visible fluorescence, fluorescence polarization, or luminescence. Additionally, the cell growth device 330 may include additional sensors for measuring, e.g., dissolved oxygen, carbon dioxide, pH, conductivity, and the like. For additional details regarding rotating growth vials and cell growth devices see U.S. Pat. No. 10,435,662, issued 8 Oct. 2019; U.S. Pat. No. 10,443,031, issued 15 Oct. 2019; and U.S. Ser. No. 16/552,981, filed 27 Aug. 2019 and Ser. No. 16/780,640, filed 3 Feb. 2020.

### The Cell Concentration Module

As described above in relation to the rotating growth vial and cell growth module, in order to obtain an adequate number of cells for transformation or transfection, cells typically are grown to a specific optical density in medium appropriate for the growth of the cells of interest; however, for effective transformation or transfection, it is desirable to decrease the volume of the cells as well as render the cells competent via buffer or medium exchange. Thus, one sub-component or module that is desired in cell processing systems to perform the methods described herein is a module or component that can grow, perform buffer exchange, and/or concentrate cells and render them competent so that they may be transformed or transfected with the nucleic acids needed for genome editing.

FIG. 4A shows a retentate member 422 (top), permeate member 420 (middle) and a tangential flow assembly 410 (bottom) comprising the retentate member 422, membrane 424 (not seen in FIG. 4A), and permeate member 420 (also not seen). In FIG. 4A, retentate member 422 comprises a tangential flow channel 402, which has a serpentine configuration that initiates at one lower corner of retentate member 422—specifically at retentate port 428—traverses across and up then down and across retentate member 422, ending in the other lower corner of retentate member 422 at a second retentate port 428. Also seen on retentate member 422 are energy directors 491, which circumscribe the region where a membrane or filter (not seen in this FIG. 4A) is seated, as well as interdigitate between areas of channel 402. Energy directors 491 in this embodiment mate with and serve to facilitate ultrasonic welding or bonding of retentate member 422 with permeate/filtrate member 420 via the energy director component 491 on permeate/filtrate member 420 (at right). Additionally, countersinks 423 can be seen, two on the bottom one at the top middle of retentate member 422. Countersinks 423 are used to couple and tangential flow assembly 410 to a reservoir assembly (not seen in this FIG. 4A but see FIG. 4B).

Permeate/filtrate member 420 is seen in the middle of FIG. 4A and comprises, in addition to energy director 491, through-holes for retentate ports 428 at each bottom corner (which mate with the through-holes for retentate ports 428 at the bottom corners of retentate member 422), as well as a tangential flow channel 402 and two permeate/filtrate ports 426 positioned at the top and center of permeate member 420. The tangential flow channel 402 structure in this embodiment has a serpentine configuration and an undulating geometry, although other geometries may be used. Permeate member 420 also comprises countersinks 423, coincident with the countersinks 423 on retentate member 420.

On the left of FIG. 4A is a tangential flow assembly 410 comprising the retentate member 422 and permeate member 420 seen in this FIG. 4A. In this view, retentate member 422 is “on top” of the view, a membrane (not seen in this view of the assembly) would be adjacent and under retentate member 422 and permeate member 420 (also not seen in this view of the assembly) is adjacent to and beneath the membrane. Again countersinks 423 are seen, where the countersinks in the retentate member 422 and the permeate member 420 are coincident and configured to mate with threads or mating elements for the countersinks disposed on a reservoir assembly (not seen in FIG. 4A but see FIG. 4B).

A membrane or filter is disposed between the retentate and permeate members, where fluids can flow through the membrane but cells cannot and are thus retained in the flow channel disposed in the retentate member. Filters or membranes appropriate for use in the TFF device/module are those that are solvent resistant, are contamination free during filtration, and are able to retain the types and sizes of cells of interest. For example, in order to retain small cell types such as bacterial cells, pore sizes can be as low as 0.2 μm, however for other cell types, the pore sizes can be as high as 20 μm. Indeed, the pore sizes useful in the TFF device/module include filters with sizes from 0.20 μm, 0.21 μm, 0.22 μm, 0.23 μm, 0.24 μm, 0.25 μm, 0.26 μm, 0.27 μm, 0.28 μm, 0.29 μm, 0.30 μm, 0.31 μm, 0.32 μm, 0.33 μm, 0.34 μm, 0.35 μm, 0.36 μm, 0.37 μm, 0.38 μm, 0.39 μm, 0.40 μm, 0.41 μm, 0.42 μm, 0.43 μm, 0.44 μm, 0.45 μm, 0.46 μm, 0.47 μm, 0.48 μm, 0.49 μm, 0.50 μm and larger. The filters may be fabricated from any suitable non-reactive material including cellulose mixed ester (cellulose nitrate and acetate) (CME), polycarbonate (PC), polyvinylidene fluoride (PVDF), polyethersulfone (PES), polytetrafluoroethylene (PTFE), nylon, glass fiber, or metal substrates as in the case of laser or electrochemical etching.

The length of the channel structure 402 may vary depending on the volume of the cell culture to be grown and the optical density of the cell culture to be concentrated. The length of the channel structure typically is from 60 mm to 300 mm, or from 70 mm to 200 mm, or from 80 mm to 100 mm. The cross-section configuration of the flow channel 402 may be round, elliptical, oval, square, rectangular, trapezoidal, or irregular. If square, rectangular, or another shape with generally straight sides, the cross section may be from about 10 μm to 1000 μm wide, or from 200 μm to 800 μm wide, or from 300 μm to 700 μm wide, or from 400 μm to 600 μm wide; and from about 10 μm to 1000 μm high, or from 200 μm to 800 μm high, or from 300 μm to 700 μm high, or from 400 μm to 600 μm high. If the cross section of the flow channel 402 is generally round, oval or elliptical, the radius of the channel may be from about 50 μm to 1000 μm in hydraulic radius, or from 5 μm to 800 μm in hydraulic radius, or from 200 μm to 700 μm in hydraulic radius, or from 300 μm to 600 μm wide in hydraulic radius, or from about 200 to 500 μm in hydraulic radius. Moreover, the volume of the channel in the retentate 422 and permeate 420 members may be different depending on the depth of the channel in each member.

FIG. 4B shows front perspective (right) and rear perspective (left) views of a reservoir assembly 450 configured to be used with the tangential flow assembly 410 seen in FIG. 4A. Seen in the front perspective view (e.g., “front” being the side of reservoir assembly 450 that is coupled to the tangential flow assembly 410 seen in FIG. 4A) are retentate reservoirs 452 on either side of permeate reservoir 454. Also seen are permeate ports 426, retentate ports 428, and three threads or mating elements 425 for countersinks 423 (countersinks 423 not seen in this FIG. 4B). Threads or mating elements 425 for countersinks 423 are configured to mate or couple the tangential flow assembly 410 (seen in FIG. 4A) to reservoir assembly 450. Alternatively or in addition, fasteners, sonic welding or heat stakes may be used to mate or couple the tangential flow assembly 410 to reservoir assembly 450. In addition gasket 445 is seen covering the top of reservoir assembly 450. Gasket 445 is described in detail in relation to FIG. 4E. At left in FIG. 4B is a rear perspective view of reservoir assembly 1250, where “rear” is the side of reservoir assembly 450 that is not coupled to the tangential flow assembly. Seen are retentate reservoirs 452, permeate reservoir 454, and gasket 445.

The TFF device may be fabricated from any robust material in which channels (and channel branches) may be milled including stainless steel, silicon, glass, aluminum, or plastics including cyclic-olefin copolymer (COC), cyclo-olefin polymer (COP), polystyrene, polyvinyl chloride, polyethylene, polyamide, polyethylene, polypropylene, acrylonitrile butadiene, polycarbonate, polyetheretheketone (PEEK), poly(methyl methylacrylate) (PMMA), polysulfone, and polyurethane, and co-polymers of these and other polymers. If the TFF device/module is disposable, preferably it is made of plastic. In some embodiments, the material used to fabricate the TFF device/module is thermally-conductive so that the cell culture may be heated or cooled to a desired temperature. In certain embodiments, the TFF device is formed by precision mechanical machining, laser machining, electro discharge machining (for metal devices); wet or dry etching (for silicon devices); dry or wet etching, powder or sandblasting, photostructuring (for glass devices); or thermoforming, injection molding, hot embossing, or laser machining (for plastic devices) using the materials mentioned above that are amenable to this mass production techniques.

FIG. 4C depicts a top-down view of the reservoir assemblies 450 shown in FIG. 4B. FIG. 4D depicts a cover 444 for reservoir assembly 450 shown in FIGS. 4B and 4E depicts a gasket 445 that in operation is disposed on cover 444 of reservoir assemblies 450 shown in FIG. 4B. FIG. 4C is a top-down view of reservoir assembly 450, showing the tops of the two retentate reservoirs 452, one on either side of permeate reservoir 454. Also seen are grooves 432 that will mate with a pneumatic port (not shown), and fluid channels 434 that reside at the bottom of retentate reservoirs 452, which fluidically couple the retentate reservoirs 452 with the retentate ports 428 (not shown), via the through-holes for the retentate ports in permeate member 420 and membrane 424 (also not shown). FIG. 4D depicts a cover 444 that is configured to be disposed upon the top of reservoir assembly 450. Cover 444 has round cut-outs at the top of retentate reservoirs 452 and permeate/filtrate reservoir 454. Again, at the bottom of retentate reservoirs 452 fluid channels 434 can be seen, where fluid channels 434 fluidically couple retentate reservoirs 452 with the retentate ports 428 (not shown). Also shown are three pneumatic ports 430 for each retentate reservoir 452 and permeate/filtrate reservoir 454. FIG. 4E depicts a gasket 445 that is configures to be disposed upon the cover 444 of reservoir assembly 450. Seen are three fluid transfer ports 442 for each retentate reservoir 452 and for permeate/filtrate reservoir 454. Again, three pneumatic ports 430, for each retentate reservoir 452 and for permeate/filtrate reservoir 454, are shown.

The overall work flow for cell growth comprises loading a cell culture to be grown into a first retentate reservoir, optionally bubbling air or an appropriate gas through the cell culture, passing or flowing the cell culture through the first retentate port then tangentially through the TFF channel structure while collecting medium or buffer through one or both of the permeate ports 406, collecting the cell culture through a second retentate port 404 into a second retentate reservoir, optionally adding additional or different medium to the cell culture and optionally bubbling air or gas through the cell culture, then repeating the process, all while measuring, e.g., the optical density of the cell culture in the retentate reservoirs continuously or at desired intervals. Measurements of optical densities (OD) at programmed time intervals are accomplished using a 600 nm Light Emitting Diode (LED) that has been columnated through an optic into the retentate reservoir(s) containing the growing cells. The light continues through a collection optic to the detection system which consists of a (digital) gain-controlled silicone photodiode. Generally, optical density is shown as the absolute value of the logarithm with base 10 of the power transmission factors of an optical attenuator: OD=−log 10 (Power out/Power in). Since OD is the measure of optical attenuation—that is, the sum of absorption, scattering, and reflection—the TFF device OD measurement records the overall power transmission, so as the cells grow and become denser in population, the OD (the loss of signal) increases. The OD system is pre-calibrated against OD standards with these values stored in an on-board memory accessible by the measurement program.

In the channel structure, the membrane bifurcating the flow channels retains the cells on one side of the membrane (the retentate side 422) and allows unwanted medium or buffer to flow across the membrane into a filtrate or permeate side (e.g., permeate member 420) of the device. Bubbling air or other appropriate gas through the cell culture both aerates and mixes the culture to enhance cell growth. During the process, medium that is removed during the flow through the channel structure is removed through the permeate/filtrate ports 406. Alternatively, cells can be grown in one reservoir with bubbling or agitation without passing the cells through the TFF channel from one reservoir to the other.

The overall work flow for cell concentration using the TFF device/module involves flowing a cell culture or cell sample tangentially through the channel structure. As with the cell growth process, the membrane bifurcating the flow channels retains the cells on one side of the membrane and allows unwanted medium or buffer to flow across the membrane into a permeate/filtrate side (e.g., permeate member 420) of the device. In this process, a fixed volume of cells in medium or buffer is driven through the device until the cell sample is collected into one of the retentate ports 404, and the medium/buffer that has passed through the membrane is collected through one or both of the permeate/filtrate ports 406. All types of prokaryotic and eukaryotic cells—both adherent and non-adherent cells—can be grown in the TFF device. Adherent cells may be grown on beads or other cell scaffolds suspended in medium that flow through the TFF device.

The medium or buffer used to suspend the cells in the cell concentration device/module may be any suitable medium or buffer for the type of cells being transformed or transfected, such as LB, SOC, TPD, YPG, YPAD, MEM, DMEM, IMDM, RPMI, Hanks', PBS and Ringer's solution, where the media may be provided in a reagent cartridge as part of a kit. For culture of adherent cells, cells may be disposed on beads, microcarriers, or other type of scaffold suspended in medium. Most normal mammalian tissue-derived cells—except those derived from the hematopoietic system are anchorage dependent and need a surface or cell culture support for normal proliferation. In the rotating growth vial described herein, microcarrier technology is leveraged. Microcarriers of particular use typically have a diameter of 100-300 μm and have a density slightly greater than that of the culture medium (thus facilitating an easy separation of cells and medium for, e.g., medium exchange) yet the density must also be sufficiently low to allow complete suspension of the carriers at a minimum stirring rate in order to avoid hydrodynamic damage to the cells. Many different types of microcarriers are available, and different microcarriers are optimized for different types of cells. There are positively charged carriers, such as Cytodex 1 (dextran-based, GE Healthcare), DE-52 (cellulose-based, Sigma-Aldrich Labware), DE-53 (cellulose-based, Sigma-Aldrich Labware), and HLX 11-170 (polystyrene-based); collagen- or ECM- (extracellular matrix) coated carriers, such as Cytodex 3 (dextran-based, GE Healthcare) or HyQ-sphere Pro-F 102-4 (polystyrene-based, Thermo Scientific); non-charged carriers, like HyQ-sphere P 102-4 (Thermo Scientific); or macroporous carriers based on gelatin (Cultisphere, Percell Biolytica) or cellulose (Cytopore, GE Healthcare).

In both the cell growth and concentration processes, passing the cell sample through the TFF device and collecting the cells in one of the retentate ports 404 while collecting the medium in one of the permeate/filtrate ports 406 is considered “one pass” of the cell sample. The transfer between retentate reservoirs “flips” the culture. The retentate and permeate ports collecting the cells and medium, respectively, for a given pass reside on the same end of TFF device/module with fluidic connections arranged so that there are two distinct flow layers for the retentate and permeate/filtrate sides, but if the retentate port 404 resides on the retentate member of device/module (that is, the cells are driven through the channel above the membrane and the filtrate (medium) passes to the portion of the channel below the membrane), the permeate/filtrate port 406 will reside on the permeate member of device/module and vice versa (that is, if the cell sample is driven through the channel below the membrane, the filtrate (medium) passes to the portion of the channel above the membrane). Due to the high pressures used to transfer the cell culture and fluids through the flow channel of the TFF device, the effect of gravity is negligible.

At the conclusion of a “pass” in either of the growth and concentration processes, the cell sample is collected by passing through the retentate port 404 and into the retentate reservoir (not shown). To initiate another “pass”, the cell sample is passed again through the TFF device, this time in a flow direction that is reversed from the first pass. The cell sample is collected by passing through the retentate port 404 and into retentate reservoir (not shown) on the opposite end of the device/module from the retentate port 404 that was used to collect cells during the first pass. Likewise, the medium/buffer that passes through the membrane on the second pass is collected through the permeate port 406 on the opposite end of the device/module from the permeate port 406 that was used to collect the filtrate during the first pass, or through both ports. This alternating process of passing the retentate (the concentrated cell sample) through the device/module is repeated until the cells have been grown to a desired optical density, and/or concentrated to a desired volume, and both permeate ports (i.e., if there are more than one) can be open during the passes to reduce operating time. In addition, buffer exchange may be effected by adding a desired buffer (or fresh medium) to the cell sample in the retentate reservoir, before initiating another “pass”, and repeating this process until the old medium or buffer is diluted and filtered out and the cells reside in fresh medium or buffer. Note that buffer exchange and cell growth may (and typically do) take place simultaneously, and buffer exchange and cell concentration may (and typically do) take place simultaneously. For further information and alternative embodiments on TFFs see, e.g., U.S. Ser. No. 62/728,365, filed 7 Sep. 2018; 62/857,599, filed 5 Jun. 2019; and 62/867,415, filed 27 Jun. 2019.

### The Cell Transformation Module

FIG. 5A depicts an exemplary combination reagent cartridge and electroporation device 500 (“cartridge”) that may be used in an automated multi-module cell processing instrument along with the TFF module. In addition, in certain embodiments the material used to fabricate the cartridge is thermally-conductive, as in certain embodiments the cartridge 500 contacts a thermal device (not shown), such as a Peltier device or thermoelectric cooler, that heats or cools reagents in the reagent reservoirs or reservoirs 504. Reagent reservoirs or reservoirs 504 may be reservoirs into which individual tubes of reagents are inserted as shown in FIG. 5A, or the reagent reservoirs may hold the reagents without inserted tubes. Additionally, the reservoirs in a reagent cartridge may be configured for any combination of tubes, co-joined tubes, and direct-fill of reagents.

In one embodiment, the reagent reservoirs or reservoirs 504 of reagent cartridge 500 are configured to hold various size tubes, including, e.g., 250 ml tubes, 25 ml tubes, 10 ml tubes, 5 ml tubes, and Eppendorf or microcentrifuge tubes. In yet another embodiment, all reservoirs may be configured to hold the same size tube, e.g., 5 ml tubes, and reservoir inserts may be used to accommodate smaller tubes in the reagent reservoir. In yet another embodiment—particularly in an embodiment where the reagent cartridge is disposable—the reagent reservoirs hold reagents without inserted tubes. In this disposable embodiment, the reagent cartridge may be part of a kit, where the reagent cartridge is pre-filled with reagents and the receptacles or reservoirs sealed with, e.g., foil, heat seal acrylic or the like and presented to a consumer where the reagent cartridge can then be used in an automated multi-module cell processing instrument. As one of ordinary skill in the art will appreciate given the present disclosure, the reagents contained in the reagent cartridge will vary depending on work flow; that is, the reagents will vary depending on the processes to which the cells are subjected in the automated multi-module cell processing instrument, e.g., protein production, cell transformation and culture, genome editing, etc.

Reagents such as cell samples, enzymes, buffers, nucleic acid vectors, editing plasmids, editing cassettes, proteins or peptides, reaction components (such as, e.g., MgCl₂, dNTPs, nucleic acid assembly reagents, gap repair reagents, and the like), wash solutions, ethanol, and magnetic beads for nucleic acid purification and isolation, etc. may be positioned in the reagent cartridge at a known position. In some embodiments of cartridge 500, the cartridge comprises a script (not shown) readable by a processor (not shown) for dispensing the reagents. Also, the cartridge 500 as one component in an automated multi-module cell processing instrument may comprise a script specifying two, three, four, five, ten or more processes to be performed by the automated multi-module cell processing instrument. In certain embodiments, the reagent cartridge is disposable and is pre-packaged with reagents tailored to performing specific cell processing protocols, e.g., genome editing, or protein production. Because the reagent cartridge contents vary while components/modules of the automated multi-module cell processing instrument or system may not, the script associated with a particular reagent cartridge matches the reagents used and cell processes performed. Thus, e.g., reagent cartridges may be pre-packaged with reagents for genome editing and a script that specifies the process steps for performing genome editing in an automated multi-module cell processing instrument, or, e.g., reagents for protein expression and a script that specifies the process steps for performing protein expression in an automated multi-module cell processing instrument.

For example, the reagent cartridge may comprise a script to pipette competent cells from a reservoir, transfer the cells to a transformation module, pipette a nucleic acid solution comprising a vector with an editing cassette from another reservoir in the reagent cartridge, transfer the nucleic acid solution to the transformation module, initiate the transformation process for a specified time, then move the transformed cells to yet another reservoir in the reagent cassette or to another module such as a cell growth module in the automated multi-module cell processing instrument. In another example, the reagent cartridge may comprise a script to transfer a nucleic acid solution comprising a vector from a reservoir in the reagent cassette, nucleic acid solution comprising editing cassettes in a reservoir in the reagent cassette, and a nucleic acid assembly mix from another reservoir to the nucleic acid assembly/desalting module, if present. The script may also specify process steps performed by other modules in the automated multi-module cell processing instrument. For example, the script may specify that the nucleic acid assembly/desalting reservoir be heated to 50° C. for 30 min to generate an assembled product; and desalting and resuspension of the assembled product via magnetic bead-based nucleic acid purification involving a series of pipette transfers and mixing of magnetic beads, ethanol wash, and buffer.

As described in relation to FIGS. 5B and 5C below, the exemplary reagent cartridges for use in the automated multi-module cell processing instruments may include one or more electroporation devices, preferably flow-through electroporation (FTEP) devices. In yet other embodiments, the reagent cartridge is separate from the transformation module. Electroporation is a widely-used method for permeabilization of cell membranes that works by temporarily generating pores in the cell membranes with electrical stimulation. Applications of electroporation include the delivery of DNA, RNA, siRNA, peptides, proteins, antibodies, drugs or other substances to a variety of cells such as mammalian cells (including human cells), plant cells, archea, yeasts, other eukaryotic cells, bacteria, and other cell types. Electrical stimulation may also be used for cell fusion in the production of hybridomas or other fused cells. During a typical electroporation procedure, cells are suspended in a buffer or medium that is favorable for cell survival. For bacterial cell electroporation, low conductance mediums, such as water, glycerol solutions and the like, are often used to reduce the heat production by transient high current. In traditional electroporation devices, the cells and material to be electroporated into the cells (collectively “the cell sample”) are placed in a cuvette embedded with two flat electrodes for electrical discharge. For example, Bio-Rad (Hercules, Calif.) makes the GENE PULSER XCELL™ line of products to electroporate cells in cuvettes. Traditionally, electroporation requires high field strength; however, the flow-through electroporation devices included in the reagent cartridges achieve high efficiency cell electroporation with low toxicity. The reagent cartridges of the disclosure allow for particularly easy integration with robotic liquid handling instrumentation that is typically used in automated instruments and systems such as air displacement pipettors. Such automated instrumentation includes, but is not limited to, off-the-shelf automated liquid handling systems from Tecan (Mannedorf, Switzerland), Hamilton (Reno, Nev.), Beckman Coulter (Fort Collins, Colo.), etc.

FIGS. 5B and 5C are top perspective and bottom perspective views, respectively, of an exemplary FTEP device 550 that may be part of (e.g., a component in) reagent cartridge 500 in FIG. 5A or may be a stand-alone module; that is, not a part of a reagent cartridge or other module. FIG. 5B depicts an FTEP device 550. The FTEP device 550 has wells that define cell sample inlets 552 and cell sample outlets 554. FIG. 5C is a bottom perspective view of the FTEP device 550 of FIG. 5B. An inlet well 552 and an outlet well 554 can be seen in this view. Also seen in FIG. 5C are the bottom of an inlet 562 corresponding to well 552, the bottom of an outlet 564 corresponding to the outlet well 554, the bottom of a defined flow channel 566 and the bottom of two electrodes 568 on either side of flow channel 566. The FTEP devices may comprise push-pull pneumatic means to allow multi-pass electroporation procedures; that is, cells to electroporated may be “pulled” from the inlet toward the outlet for one pass of electroporation, then be “pushed” from the outlet end of the FTEP device toward the inlet end to pass between the electrodes again for another pass of electroporation. Further, this process may be repeated one to many times. For additional information regarding FTEP devices, see, e.g., U.S. Pat. No. 10,435,713, issued 8 Oct. 2019; U.S. Pat. No. 10,443,074, issued 15 Oct. 2019; U.S. Pat. No. 10,323,258, issued 18 Jun. 2019; U.S. Pat. No. 10,508,288, issued 17 Dec. 2019; U.S. Pat. No. 10,415,058, issued 17 Sep. 2019; and U.S. Ser. No. 16/550,790, filed 26 Aug. 2019; and Ser. No. 16/571,080, filed 14 Sep. 2019. Further, other embodiments of the reagent cartridge may provide or accommodate electroporation devices that are not configured as FTEP devices, such as those described in U.S. Ser. No. 16/109,156, filed 22 Aug. 2018. For reagent cartridges useful in the present automated multi-module cell processing instruments, see, e.g., U.S. Pat. No. 10,376,889, issued 13 Aug. 2019; U.S. Pat. No. 10,406,525, issued 10 Sep. 2019; U.S. Pat. No. 10,478,822, issued 19 Nov. 2019; U.S. Pat. No. 10,576,474, issued 3 Feb. 2020; and U.S. Ser. No. 16/749,757, filed 22 Jan. 2020.

Additional details of the FTEP devices are illustrated in FIGS. 5D-5F. Note that in the FTEP devices in FIGS. 5D-5F the electrodes are placed such that a first electrode is placed between an inlet and a narrowed region of the flow channel, and the second electrode is placed between the narrowed region of the flow channel and an outlet. FIG. 5D shows a top planar view of an FTEP device 550 having an inlet 552 for introducing a fluid containing cells and exogenous material into FTEP device 550 and an outlet 554 for removing the transformed cells from the FTEP following electroporation. The electrodes 568 are introduced through channels (not shown) in the device. FIG. 5E shows a cutaway view from the top of the FTEP device 550, with the inlet 552, outlet 554, and electrodes 568 positioned with respect to a flow channel 566. FIG. 5F shows a side cutaway view of FTEP device 550 with the inlet 552 and inlet channel 572, and outlet 554 and outlet channel 574. The electrodes 568 are positioned in electrode channels 576 so that they are in fluid communication with the flow channel 566, but not directly in the path of the cells traveling through the flow channel 566. Note that the first electrode is placed between the inlet and the narrowed region of the flow channel, and the second electrode is placed between the narrowed region of the flow channel and the outlet. The electrodes 568 in this aspect of the device are positioned in the electrode channels 576 which are generally perpendicular to the flow channel 566 such that the fluid containing the cells and exogenous material flows from the inlet channel 572 through the flow channel 566 to the outlet channel 574, and in the process fluid flows into the electrode channels 576 to be in contact with the electrodes 568. In this aspect, the inlet channel, outlet channel and electrode channels all originate from the same planar side of the device. In certain aspects, however, the electrodes may be introduced from a different planar side of the FTEP device than the inlet and outlet channels.

In the FTEP devices of the disclosure, the toxicity level of the transformation results in greater than 30% viable cells after electroporation, preferably greater than 35%, 40%, 45%, 50%, 55%, 60%, 70%, 75%, 80%, 85%, 90%, 95% or even 99% viable cells following transformation, depending on the cell type and the nucleic acids being introduced into the cells.

The housing of the FTEP device can be made from many materials depending on whether the FTEP device is to be reused, autoclaved, or is disposable, including stainless steel, silicon, glass, resin, polyvinyl chloride, polyethylene, polyamide, polystyrene, polyethylene, polypropylene, acrylonitrile butadiene, polycarbonate, polyetheretheketone (PEEK), polysulfone and polyurethane, co-polymers of these and other polymers. Similarly, the walls of the channels in the device can be made of any suitable material including silicone, resin, glass, glass fiber, polyvinyl chloride, polyethylene, polyamide, polyethylene, polypropylene, acrylonitrile butadiene, polycarbonate, polyetheretheketone (PEEK), polysulfone and polyurethane, co-polymers of these and other polymers. Preferred materials include crystal styrene, cyclo-olefin polymer (COP) and cyclic olephin co-polymers (COC), which allow the device to be formed entirely by injection molding in one piece with the exception of the electrodes and, e.g., a bottom sealing film if present.

The FTEP devices described herein (or portions of the FTEP devices) can be created or fabricated via various techniques, e.g., as entire devices or by creation of structural layers that are fused or otherwise coupled. For example, for metal FTEP devices, fabrication may include precision mechanical machining or laser machining; for silicon FTEP devices, fabrication may include dry or wet etching; for glass FTEP devices, fabrication may include dry or wet etching, powderblasting, sandblasting, or photostructuring; and for plastic FTEP devices fabrication may include thermoforming, injection molding, hot embossing, or laser machining. The components of the FTEP devices may be manufactured separately and then assembled, or certain components of the FTEP devices (or even the entire FTEP device except for the electrodes) may be manufactured (e.g., using 3D printing) or molded (e.g., using injection molding) as a single entity, with other components added after molding. For example, housing and channels may be manufactured or molded as a single entity, with the electrodes later added to form the FTEP unit. Alternatively, the FTEP device may also be formed in two or more parallel layers, e.g., a layer with the horizontal channel and filter, a layer with the vertical channels, and a layer with the inlet and outlet ports, which are manufactured and/or molded individually and assembled following manufacture.

In specific aspects, the FTEP device can be manufactured using a circuit board as a base, with the electrodes, filter and/or the flow channel formed in the desired configuration on the circuit board, and the remaining housing of the device containing, e.g., the one or more inlet and outlet channels and/or the flow channel formed as a separate layer that is then sealed onto the circuit board. The sealing of the top of the housing onto the circuit board provides the desired configuration of the different elements of the FTEP devices of the disclosure. Also, two to many FTEP devices may be manufactured on a single substrate, then separated from one another thereafter or used in parallel. In certain embodiments, the FTEP devices are reusable and, in some embodiments, the FTEP devices are disposable. In additional embodiments, the FTEP devices may be autoclavable.

The electrodes 508 can be formed from any suitable metal, such as copper, stainless steel, titanium, aluminum, brass, silver, rhodium, gold or platinum, or graphite. One preferred electrode material is alloy 303 (UNS330300) austenitic stainless steel. An applied electric field can destroy electrodes made from of metals like aluminum. If a multiple-use (i.e., non-disposable) flow-through FTEP device is desired—as opposed to a disposable, one-use flow-through FTEP device—the electrode plates can be coated with metals resistant to electrochemical corrosion. Conductive coatings like noble metals, e.g., gold, can be used to protect the electrode plates.

As mentioned, the FTEP devices may comprise push-pull pneumatic means to allow multi-pass electroporation procedures; that is, cells to electroporated may be “pulled” from the inlet toward the outlet for one pass of electroporation, then be “pushed” from the outlet end of the flow-through FTEP device toward the inlet end to pass between the electrodes again for another pass of electroporation. This process may be repeated one to many times.

Depending on the type of cells to be electroporated (e.g., bacterial, yeast, mammalian) and the configuration of the electrodes, the distance between the electrodes in the flow channel can vary widely. For example, where the flow channel decreases in width, the flow channel may narrow to between 10 μm and 5 mm, or between 25 μm and 3 mm, or between 50 μm and 2 mm, or between 75 μm and 1 mm. The distance between the electrodes in the flow channel may be between 1 mm and 10 mm, or between 2 mm and 8 mm, or between 3 mm and 7 mm, or between 4 mm and 6 mm. The overall size of the FTEP device may be from 3 cm to 15 cm in length, or 4 cm to 12 cm in length, or 4.5 cm to 10 cm in length. The overall width of the FTEP device may be from 0.5 cm to 5 cm, or from 0.75 cm to 3 cm, or from 1 cm to 2.5 cm, or from 1 cm to 1.5 cm.

The region of the flow channel that is narrowed is wide enough so that at least two cells can fit in the narrowed portion side-by-side. For example, a typical bacterial cell is 1 μm in diameter; thus, the narrowed portion of the flow channel of the FTEP device used to transform such bacterial cells will be at least 2 μm wide. In another example, if a mammalian cell is approximately 50 μm in diameter, the narrowed portion of the flow channel of the FTEP device used to transform such mammalian cells will be at least 100 μm wide. That is, the narrowed portion of the FTEP device will not physically contort or “squeeze” the cells being transformed.

In embodiments of the FTEP device where reservoirs are used to introduce cells and exogenous material into the FTEP device, the reservoirs range in volume from 100 μL to 10 mL, or from 500 μL to 75 mL, or from 1 mL to 5 mL. The flow rate in the FTEP ranges from 0.1 mL to 5 mL per minute, or from 0.5 mL to 3 mL per minute, or from 1.0 mL to 2.5 mL per minute. The pressure in the FTEP device ranges from 1-30 psi, or from 2-10 psi, or from 3-5 psi.

To avoid different field intensities between the electrodes, the electrodes should be arranged in parallel. Furthermore, the surface of the electrodes should be as smooth as possible without pin holes or peaks. Electrodes having a roughness Rz of 1 to 10 μm are preferred. In another embodiment of the invention, the flow-through electroporation device comprises at least one additional electrode which applies a ground potential to the FTEP device.

### Cell Singulation and Enrichment Device

FIG. 6A depicts a solid wall device 6050 and a workflow for singulating cells in microwells in the solid wall device. At the top left of the figure (i), there is depicted solid wall device 6050 with microwells 6052. A section 6054 of substrate 6050 is shown at (ii), also depicting microwells 6052. At (iii), a side cross-section of solid wall device 6050 is shown, and microwells 6052 have been loaded, where, in this embodiment, Poisson or substantial Poisson loading has taken place; that is, each microwell has one or no cells, and the likelihood that any one microwell has more than one cell is low. At (iv), workflow 6040 is illustrated where substrate 6050 having microwells 6052 shows microwells 6056 with one cell per microwell, microwells 6057 with no cells in the microwells, and one microwell 6060 with two cells in the microwell. In step 6051, the cells in the microwells are allowed to double approximately 2-150 times to form clonal colonies (v), then genome editing is allowed to occur 6053.

After processing 6053 (e.g., genome editing), some cells in the colonies of cells may die, e.g., by fitness effects from processing events, and there may be a lag in growth for the cells that survive but must recover following editing (microwells 6058), where cells that do not undergo editing may thrive (microwells 6059) (vi). All cells are allowed to continue grow to establish colonies and normalize, where the colonies of modified cells in microwells 6058 catch up in size and/or cell number with the cells in microwells 6059 that do not undergo editing (vii). Once the cell colonies are normalized, either pooling 6060 of all cells in the microwells can take place, in which case the cells are enriched for modified cells by eliminating the bias from non-modified cells and fitness effects; alternatively, colony growth in the microwells is monitored after processing, and slow growing colonies (e.g., the cells in microwells 6058) are identified and selected 6061 (e.g., “cherry picked”) resulting in even greater enrichment of modified cells.

In growing the cells, the medium used will depend, of course, on the type of cells being processed—e.g., bacterial, yeast or mammalian. For example, medium for yeast cell growth includes LB, SOC, TPD, YPG, YPAD, MEM and DMEM.

A module useful for performing the method depicted in FIG. 6A is a solid wall isolation, incubation, and normalization (SWIIN) module. FIG. 6B depicts an embodiment of a SWIIN module 650 from an exploded top perspective view. In SWIIN module 650 the retentate member is formed on the bottom of a top of a SWIIN module component and the permeate member is formed on the top of the bottom of a SWIIN module component.

The SWIIN module 650 in FIG. 6B comprises from the top down, a reservoir gasket or cover 658, a retentate member 604 (where a retentate flow channel cannot be seen in this FIG. 6B), a perforated member 601 swaged with a filter (filter not seen in FIG. 6B), a permeate member 608 comprising integrated reservoirs (permeate reservoirs 652 and retentate reservoirs 654), and two reservoir seals 662, which seal the bottom of permeate reservoirs 652 and retentate reservoirs 654. A permeate channel 660a can be seen disposed on the top of permeate member 608, defined by a raised portion 676 of serpentine channel 660a, and ultrasonic tabs 664 can be seen disposed on the top of permeate member 608 as well. The perforations that form the wells on perforated member 601 are not seen in this FIG. 6B; however, through-holes 666 to accommodate the ultrasonic tabs 664 are seen. In addition, supports 670 are disposed at either end of SWIIN module 650 to support SWIIN module 650 and to elevate permeate member 608 and retentate member 604 above reservoirs 652 and 654 to minimize bubbles or air entering the fluid path from the permeate reservoir to serpentine channel 660a or the fluid path from the retentate reservoir to serpentine channel 660b (neither fluid path is seen in this FIG. 6B).

In this FIG. 6B, it can be seen that the serpentine channel 660a that is disposed on the top of permeate member 608 traverses permeate member 608 for most of the length of permeate member 608 except for the portion of permeate member 608 that comprises permeate reservoirs 652 and retentate reservoirs 654 and for most of the width of permeate member 608. As used herein with respect to the distribution channels in the retentate member or permeate member, “most of the length” means about 95% of the length of the retentate member or permeate member, or about 90%, 85%, 80%, 75%, or 70% of the length of the retentate member or permeate member. As used herein with respect to the distribution channels in the retentate member or permeate member, “most of the width” means about 95% of the width of the retentate member or permeate member, or about 90%, 85%, 80%, 75%, or 70% of the width of the retentate member or permeate member.

In this embodiment of a SWIIN module, the perforated member includes through-holes to accommodate ultrasonic tabs disposed on the permeate member. Thus, in this embodiment the perforated member is fabricated from 316 stainless steel, and the perforations form the walls of microwells while a filter or membrane is used to form the bottom of the microwells. Typically, the perforations (microwells) are approximately 150 μm-200 μm in diameter, and the perforated member is approximately 125 μm deep, resulting in microwells having a volume of approximately 2.5 nl, with a total of approximately 200,000 microwells. The distance between the microwells is approximately 279 μm center-to-center. Though here the microwells have a volume of approximately 2.5 nl, the volume of the microwells may be from 1 to 25 nl, or preferably from 2 to 10 nl, and even more preferably from 2 to 4 nl. As for the filter or membrane, like the filter described previously, filters appropriate for use are solvent resistant, contamination free during filtration, and are able to retain the types and sizes of cells of interest. For example, in order to retain small cell types such as bacterial cells, pore sizes can be as low as 0.10 μm, however for other cell types (e.g., such as for mammalian cells), the pore sizes can be as high as 10.0 μm-20.0 μm or more. Indeed, the pore sizes useful in the cell concentration device/module include filters with sizes from 0.10 μm, 0.11 μm, 0.12 μm, 0.13 μm, 0.14 μm, 0.15 μm, 0.16 μm, 0.17 μm, 0.18 μm, 0.19 μm, 0.20 μm, 0.21 μm, 0.22 μm, 0.23 μm, 0.24 μm, 0.25 μm, 0.26 μm, 0.27 μm, 0.28 μm, 0.29 μm, 0.30 μm, 0.31 μm, 0.32 μm, 0.33 μm, 0.34 μm, 0.35 μm, 0.36 μm, 0.37 μm, 0.38 μm, 0.39 μm, 0.40 μm, 0.41 μm, 0.42 μm, 0.43 μm, 0.44 μm, 0.45 μm, 0.46 μm, 0.47 μm, 0.48 μm, 0.49 μm, 0.50 μm and larger. The filters may be fabricated from any suitable material including cellulose mixed ester (cellulose nitrate and acetate) (CME), polycarbonate (PC), polyvinylidene fluoride (PVDF), polyethersulfone (PES), polytetrafluoroethylene (PTFE), nylon, or glass fiber.

The cross-section configuration of the mated serpentine channel may be round, elliptical, oval, square, rectangular, trapezoidal, or irregular. If square, rectangular, or another shape with generally straight sides, the cross section may be from about 2 mm to 15 mm wide, or from 3 mm to 12 mm wide, or from 5 mm to 10 mm wide. If the cross section of the mated serpentine channel is generally round, oval or elliptical, the radius of the channel may be from about 3 mm to 20 mm in hydraulic radius, or from 5 mm to 15 mm in hydraulic radius, or from 8 mm to 12 mm in hydraulic radius.

Serpentine channels 660a and 660b can have approximately the same volume or a different volume. For example, each “side” or portion 660a, 660b of the serpentine channel may have a volume of, e.g., 2 mL, or serpentine channel 660a of permeate member 608 may have a volume of 2 mL, and the serpentine channel 660b of retentate member 604 may have a volume of, e.g., 3 mL. The volume of fluid in the serpentine channel may range from about 2 mL to about 80 mL, or about 4 mL to 60 mL, or from 5 mL to 40 mL, or from 6 mL to 20 mL (note these volumes apply to a SWIIN module comprising a, e.g., 50-500K perforation member). The volume of the reservoirs may range from 5 mL to 50 mL, or from 7 mL to 40 mL, or from 8 mL to 30 mL or from 10 mL to 20 mL, and the volumes of all reservoirs may be the same or the volumes of the reservoirs may differ (e.g., the volume of the permeate reservoirs is greater than that of the retentate reservoirs).

The serpentine channel portions 660a and 660b of the permeate member 608 and retentate member 604, respectively, are approximately 200 mm long, 130 mm wide, and 4 mm thick, though in other embodiments, the retentate and permeate members can be from 75 mm to 400 mm in length, or from 100 mm to 300 mm in length, or from 150 mm to 250 mm in length; from 50 mm to 250 mm in width, or from 75 mm to 200 mm in width, or from 100 mm to 150 mm in width; and from 2 mm to 15 mm in thickness, or from 4 mm to 10 mm in thickness, or from 5 mm to 8 mm in thickness. Embodiments the retentate (and permeate) members may be fabricated from PMMA (poly(methyl methacrylate) or other materials may be used, including polycarbonate, cyclic olefin copolymer (COC), glass, polyvinyl chloride, polyethylene, polyamide, polypropylene, polysulfone, polyurethane, and co-polymers of these and other polymers. Preferably at least the retentate member is fabricated from a transparent material so that the cells can be visualized (see, e.g., FIG. 6E and the description thereof). For example, a video camera may be used to monitor cell growth by, e.g., density change measurements based on an image of an empty well, with phase contrast, or if, e.g., a chromogenic marker, such as a chromogenic protein, is used to add a distinguishable color to the cells. Chromogenic markers such as blitzen blue, dreidel teal, virginia violet, vixen purple, prancer purple, tinsel purple, maccabee purple, donner magenta, cupid pink, seraphina pink, scrooge orange, and leor orange (the Chromogenic Protein Paintbox, all available from ATUM (Newark, Calif.)) obviate the need to use fluorescence, although fluorescent cell markers, fluorescent proteins, and chemiluminescent cell markers may also be used.

Because the retentate member preferably is transparent, colony growth in the SWIIN module can be monitored by automated devices such as those sold by JoVE (ScanLag™ system, Cambridge, Mass.) (also see Levin-Reisman, et al., Nature Methods, 7:737-39 (2010)). Cell growth for, e.g., mammalian cells may be monitored by, e.g., the growth monitor sold by IncuCyte (Ann Arbor, Mich.) (see also, Choudhry, PLos One, 11(2):e0148469 (2016)). Further, automated colony pickers may be employed, such as those sold by, e.g., TECAN (Pickolo™ system, Mannedorf, Switzerland); Hudson Inc. (RapidPick™, Springfield, N.J.); Molecular Devices (QPix 400™ system, San Jose, Calif.); and Singer Instruments (PIXL system, Somerset, UK).

Due to the heating and cooling of the SWIIN module, condensation may accumulate on the retentate member which may interfere with accurate visualization of the growing cell colonies. Condensation of the SWIIN module 650 may be controlled by, e.g., moving heated air over the top of (e.g., retentate member) of the SWIIN module 650, or by applying a transparent heated lid over at least the serpentine channel portion 660b of the retentate member 604. See, e.g., FIG. 6E and the description thereof infra.

In SWIIN module 650 cells and medium—at a dilution appropriate for Poisson or substantial Poisson distribution of the cells in the microwells of the perforated member—are flowed into serpentine channel 660b from ports in retentate member 604, and the cells settle in the microwells while the medium passes through the filter into serpentine channel 660a in permeate member 608. The cells are retained in the microwells of perforated member 601 as the cells cannot travel through filter 603. Appropriate medium may be introduced into permeate member 608 through permeate ports 611. The medium flows upward through filter 603 to nourish the cells in the microwells (perforations) of perforated member 601. Additionally, buffer exchange can be effected by cycling medium through the retentate and permeate members. In operation, the cells are deposited into the microwells, are grown for an initial, e.g., 2-100 doublings, and in certain embodiments, genome editing may be induced by, e.g., raising or lower the temperature of the SWIIN to induce a temperature inducible promoter, or by removing growth medium from the permeate member and replacing the growth medium with a medium comprising a chemical component that induces an inducible promoter. In certain other embodiments, no induction as necessary, as relevant components of the nuclease-mediated editing system may be under the control of constitutive promoters.

Once editing has taken place, in certain embodiments, the temperature of the SWIIN may be modified to stop editing, or the inducing medium may be removed and replaced with fresh medium lacking the chemical component thereby de-activating inducible promoters. For example, in certain embodiments, increasing the temperature of the SWINN to 30° C. may halt or slow down editing. The modified cells then continue to grow in the SWIIN module 650 until the growth of the cell colonies in the microwells is normalized. For the normalization protocol, once the colonies are normalized, the colonies are flushed from the microwells by applying fluid or air pressure (or both) to the permeate member serpentine channel 660a and thus to filter 603 and pooled. Alternatively, if cherry picking is desired, the growth of the cell colonies in the microwells is monitored, and slow-growing colonies are directly selected; or, fast-growing colonies are eliminated.

FIG. 6C is a top perspective view of a SWIIN module with the retentate and perforated members in partial cross section. In this FIG. 6C, it can be seen that serpentine channel 660a is disposed on the top of permeate member 608 is defined by raised portions 676 and traverses permeate member 608 for most of the length and width of permeate member 608 except for the portion of permeate member 608 that comprises the permeate and retentate reservoirs (note only one retentate reservoir 652 can be seen). Moving from left to right, reservoir gasket 658 is disposed upon the integrated reservoir cover 678 (cover not seen in this FIG. 6C) of retentate member 604. Gasket 658 comprises reservoir access apertures 632a, 632b, 632c, and 632d, as well as pneumatic ports 633a, 633b, 633c and 633d. Also at the far left end is support 670. Disposed under permeate reservoir 652 can be seen one of two reservoir seals 662. In addition to the retentate member being in cross section, the perforated member 601 and filter 603 (filter 603 is not seen in this FIG. 6C) are in cross section. Note that there are a number of ultrasonic tabs 664 disposed at the right end of SWIIN module 650 and on raised portion 676 which defines the channel turns of serpentine channel 660a, including ultrasonic tabs 664 extending through through-holes 666 of perforated member 601. There is also a support 670 at the end distal reservoirs 652, 654 of permeate member 608.

FIG. 6D is a side perspective view of an assembled SWIIIN module 650, including, from right to left, reservoir gasket 658 disposed upon integrated reservoir cover 678 (not seen) of retentate member 604. Gasket 658 may be fabricated from rubber, silicone, nitrile rubber, polytetrafluoroethylene, a plastic polymer such as polychlorotrifluoroethylene, or other flexible, compressible material. Gasket 658 comprises reservoir access apertures 632a, 632b, 632c, and 632d, as well as pneumatic ports 633a, 633b, 633c and 633d. Also at the far-left end is support 670 of permeate member 608. In addition, permeate reservoir 652 can be seen, as well as one reservoir seal 662. At the far-right end is a second support 670.

Imaging of cell colonies growing in the wells of the SWIIN is desired in most implementations for, e.g., monitoring both cell growth and device performance and imaging is necessary for cherry-picking implementations. Real-time monitoring of cell growth in the SWIIN requires backlighting, retentate plate (top plate) condensation management and a system-level approach to temperature control, air flow, and thermal management. In some implementations, imaging employs a camera or CCD device with sufficient resolution to be able to image individual wells. For example, in some configurations a camera with a 9-pixel pitch is used (that is, there are 9 pixels center-to-center for each well). Processing the images may, in some implementations, utilize reading the images in grayscale, rating each pixel from low to high, where wells with no cells will be brightest (due to full or nearly-full light transmission from the backlight) and wells with cells will be dim (due to cells blocking light transmission from the backlight). After processing the images, thresholding is performed to determine which pixels will be called “bright” or “dim”, spot finding is performed to find bright pixels and arrange them into blocks, and then the spots are arranged on a hexagonal grid of pixels that correspond to the spots. Once arranged, the measure of intensity of each well is extracted, by, e.g., looking at one or more pixels in the middle of the spot, looking at several to many pixels at random or pre-set positions, or averaging X number of pixels in the spot. In addition, background intensity may be subtracted. Thresholding is again used to call each well positive (e.g., containing cells) or negative (e.g., no cells in the well). The imaging information may be used in several ways, including taking images at time points for monitoring cell growth. Monitoring cell growth can be used to, e.g., remove the “muffin tops” of fast-growing cells followed by removal of all cells or removal of cells in “rounds” as described above, or recover cells from specific wells (e.g., slow-growing cell colonies); alternatively, wells containing fast-growing cells can be identified and areas of UV light covering the fast-growing cell colonies can be projected (or rastered with shutters) onto the SWIIN to irradiate or inhibit growth of those cells. Imaging may also be used to assure proper fluid flow in the serpentine channel 660.

FIG. 6E depicts the embodiment of the SWIIN module in FIGS. 6B-6D further comprising a heat management system including a heater and a heated cover. The heater cover facilitates the condensation management that is required for imaging. Assembly 698 comprises a SWIIN module 650 seen lengthwise in cross section, where one permeate reservoir 652 is seen. Disposed immediately upon SWIIN module 650 is cover 694 and disposed immediately below SWIIN module 650 is backlight 680, which allows for imaging. Beneath and adjacent to the backlight and SWIIN module is insulation 682, which is disposed over a heatsink 684. In this FIG. 6E, the fins of the heatsink would be in-out of the page. In addition there is also axial fan 686 and heat sink 688, as well as two thermoelectric coolers 692, and a controller 690 to control the pneumatics, thermoelectric coolers, fan, solenoid valves, etc. The arrows denote cool air coming into the unit and hot air being removed from the unit. It should be noted that control of heating allows for growth of many different types of cells (prokaryotic and eukaryotic) as well as strains of cells that are, e.g., temperature sensitive, etc., and allows use of temperature-sensitive promoters. Temperature control allows for protocols to be adjusted to account for differences in transformation efficiency, cell growth and viability. For more details regarding solid wall isolation incubation and normalization devices see U.S. Ser. No. 16/399,988, filed 30 Apr. 2019; Ser. No. 16/454,865, filed 26 Jun. 2019; and Ser. No. 16/540,606, filed 14 Aug. 2019. For alternative isolation, incubation and normalization modules, see U.S. Ser. No. 16/536,049, filed 8 Aug. 2019.

### Use of the Automated Multi-Module Cell Processing Instrument

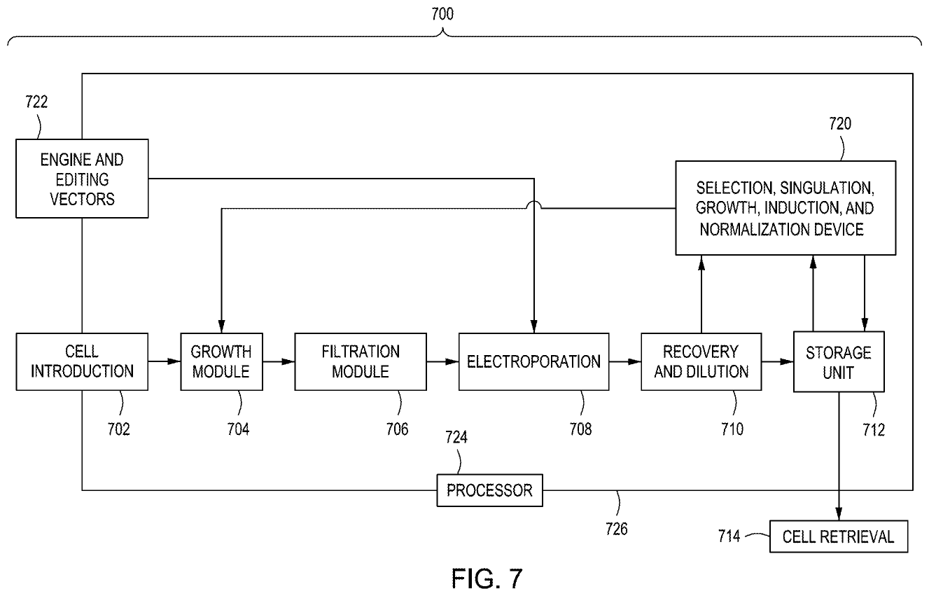

FIG. 7 illustrates an embodiment of a multi-module cell processing instrument. This embodiment depicts an exemplary system that can be utilized to perform automated genome editing on, e.g., a microbial cell population. The cell processing instrument 700 may include a housing 726, a reservoir for storing cells to be transformed or transfected 702, and a cell growth module (comprising, e.g., a rotating growth vial) 704. The cells to be transformed are transferred from a reservoir 702 to the cell growth module 704 to be cultured until the cells hit a target OD. Once the cells hit the target OD, the growth module may cool or freeze the cells for later processing or transfer the cells to a cell concentration (e.g., filtration) module 706 where the cells are subjected to buffer exchange and rendered electrocompetent and the volume of the cells may be reduced substantially. Once the cells have been concentrated to an appropriate volume, the cells are transferred to electroporation device 708 or other transformation module. In addition to the reservoir for storing cells 702, the multi-module cell processing instrument includes a reservoir for storing the engine vectors or combined engine/integration vectors or vectors and proteins to be introduced into the electrocompetent cell population 722. The vector backbones and editing cassettes are transferred to the electroporation device 708, which already contains the cell culture grown to a target OD. In the electroporation device 708, the nucleic acids (or nucleic acids and proteins) are electroporated into the cells. Following electroporation, the cells are transferred into an optional recovery and dilution module 710, where the cells recover briefly post-transformation.

After recovery, the cells may be transferred to a storage module 712, where the cells can be stored at, e.g., 4° C. or −20° C. for later processing, or the cells may be diluted and transferred to a SWIIN module 720. In the SWIIN 720, the cells are arrayed such that there is an average of one to twenty or fifty or so cells per microwell. The arrayed cells may be in selection medium to select for cells that have been transformed or transfected with the integration and/or engine vector(s). Once singulated, the cells grow through 2-50 doublings and establish colonies. Once colonies are established, genome editing is allowed to proceed by providing conditions (e.g., temperature) to facilitate, and in certain embodiments, induce, such editing. The modified cells are allowed to grow to terminal size (e.g., normalization of the colonies) in the microwells and may then be treated to conditions that cure the integration vector in the samples. Once cured, the cells can be flushed out of the microwells and pooled, then transferred to the storage (or recovery) unit 712 or can be transferred back to the growth module 704 for another round of processing. In between pooling and transfer to a growth module, there typically is one or more additional steps, such as cell recovery, medium exchange (rendering the cells electrocompetent), cell concentration (typically concurrently with medium exchange by, e.g., filtration. Note that the selection/singulation/growth/induction/normalization and curing modules may be the same module, where all processes are performed in, e.g., a solid wall device, or selection and/or dilution may take place in a separate vessel before the cells are transferred to the solid wall SWIIN. Similarly, the cells may be pooled after normalization, transferred to a separate vessel, and cured in the separate vessel. Once the modified cells are pooled, they may be subjected to further processing, including another round of editing.

The multi-module cell processing instrument exemplified in FIG. 7 is controlled by a processor 724 configured to operate the instrument based on user input or is controlled by one or more scripts including at least one script associated with the reagent cartridge. The processor 724 may control the timing, duration, and temperature of various processes, the dispensing of reagents, and other operations of the various modules of the instrument 700. For example, a script or the processor may control the dispensing of cells, reagents, vectors, and editing cassettes; which editing cassettes and vectors are used and in what order; the time, temperature and other conditions used in the recovery and expression module, the wavelength at which OD is read in the cell growth module, the target OD to which the cells are grown, and the target time at which the cells will reach the target OD. In addition, the processor may be programmed to notify a user (e.g., via an application) as to the progress of the cells in the automated multi-module cell processing instrument.

It should be apparent to one of ordinary skill in the art given the present disclosure that the process described may be recursive and multiplex; that is, cells may go through the workflow described in relation to FIG. 7, then the resulting culture may go through another (or several or many) rounds of additional processing (e.g., editing) with different cassettes/vectors.

### EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

### Example I. Fully-Automated Singleplex RGN-Directed Editing Run

Singleplex automated genomic editing using MAD7 nuclease was successfully performed with an automated multi-module instrument of the disclosure. For examples of multi-module cell editing instruments, see U.S. Pat. No. 10,253,316, issued 9 Apr. 2019; U.S. Pat. No. 10,329,559, issued 25 Jun. 2019; U.S. Pat. No. 10,323,242, issued 18 Jun. 2019; U.S. Pat. No. 10,421,959, issued 24 Sep. 2019; U.S. Pat. No. 10,465,185, issued 5 Nov. 2019; U.S. Pat. No. 10,519,437, issued 31 Dec. 2019; U.S. Pat. No. 10,584,333, issued 10 Mar. 2020; U.S. Pat. No. 10,584,334, issued 10 Mar. 2020; U.S. Pat. No. 10,647,982, issued 12 May 2020; U.S. Pat. No. 10,689,645, issued 23 Jun. 2020; U.S. Pat. No. 10,738,301, issued 11 Aug. 2020; and U.S. Ser. No. 16/920,853, filed 6 Jul. 2020; and Ser. No. 16/988,694, filed 9 Aug. 2020, all of which are herein incorporated by reference in their entirety.

An ampR plasmid backbone and a lacZ_F172* editing cassette were assembled via Gibson Assembly® into an “editing vector” in an isothermal nucleic acid assembly module included in the automated instrument. lacZ_F172 functionally knocks out the lacZ gene. “lacZ_F172*” indicates that the edit happens at the 172nd residue in the lacZ amino acid sequence. Following assembly, the product was de-salted in the isothermal nucleic acid assembly module using AMPure beads, washed with 80% ethanol, and eluted in buffer. The assembled editing vector and recombineering-ready, electrocompetent E. Coli cells were transferred into a transformation module for electroporation. The cells and nucleic acids were combined and allowed to mix for 1 minute, and electroporation was performed for 30 seconds. The parameters for the poring pulse were: voltage, 2400 V; length, 5 ms; interval, 50 ms; number of pulses, 1; polarity, +. The parameters for the transfer pulses were: Voltage, 150 V; length, 50 ms; interval, 50 ms; number of pulses, 20; polarity, +/−. Following electroporation, the cells were transferred to a recovery module (another growth module), and allowed to recover in SOC medium containing chloramphenicol. Carbenicillin was added to the medium after 1 hour, and the cells were allowed to recover for another 2 hours. After recovery, the cells were held at 4° C. until recovered by the user.

After the automated process and recovery, an aliquot of cells was plated on MacConkey agar base supplemented with lactose (as the sugar substrate), chloramphenicol and carbenicillin and grown until colonies appeared. White colonies represented functionally edited cells, purple colonies represented un-edited cells. All liquid transfers were performed by the automated liquid handling device of the automated multi-module cell processing instrument.

The result of the automated processing was that approximately 1.0E⁰³ total cells were transformed (comparable to conventional benchtop results), and the editing efficiency was 83.5%. The lacZ_172 edit in the white colonies was confirmed by sequencing of the edited region of the genome of the cells. Further, steps of the automated cell processing were observed remotely by webcam and text messages were sent to update the status of the automated processing procedure.

### Example II: Fully-Automated Recursive Editing Run

Recursive editing was successfully achieved using the automated multi-module cell processing system. An ampR plasmid backbone and a lacZ_V10* editing cassette were assembled via Gibson Assembly® into an “editing vector” in an isothermal nucleic acid assembly module included in the automated system. Similar to the lacZ_F172 edit, the lacZ_V10 edit functionally knocks out the lacZ gene. “lacZ_V10” indicates that the edit happens at amino acid position 10 in the lacZ amino acid sequence. Following assembly, the product was de-salted in the isothermal nucleic acid assembly module using AMPure beads, washed with 80% ethanol, and eluted in buffer. The first assembled editing vector and the recombineering-ready electrocompetent E. coli cells were transferred into a transformation module for electroporation. The cells and nucleic acids were combined and allowed to mix for 1 minute, and electroporation was performed for 30 seconds. The parameters for the poring pulse were: voltage, 2400 V; length, 5 ms; interval, 50 ms; number of pulses, 1; polarity, +. The parameters for the transfer pulses were: Voltage, 150 V; length, 50 ms; interval, 50 ms; number of pulses, 20; polarity, +/−. Following electroporation, the cells were transferred to a recovery module (another growth module) allowed to recover in SOC medium containing chloramphenicol. Carbenicillin was added to the medium after 1 hour, and the cells were grown for another 2 hours. The cells were then transferred to a centrifuge module and a media exchange was then performed. Cells were resuspended in TB containing chloramphenicol and carbenicillin where the cells were grown to OD600 of 2.7, then concentrated and rendered electrocompetent.

During cell growth, a second editing vector was prepared in an isothermal nucleic acid assembly module. The second editing vector comprised a kanamycin resistance gene, and the editing cassette comprised a galK Y145* edit. If successful, the galK Y145* edit confers on the cells the ability to uptake and metabolize galactose. The edit generated by the galK Y145* cassette introduces a stop codon at the 154th amino acid reside, changing the tyrosine amino acid to a stop codon. This edit makes the galK gene product non-functional and inhibits the cells from being able to metabolize galactose. Following assembly, the second editing vector product was de-salted in the isothermal nucleic acid assembly module using AMPure beads, washed with 80% ethanol, and eluted in buffer. The assembled second editing vector and the electrocompetent E. coli cells (that were transformed with and selected for the first editing vector) were transferred into a transformation module for electroporation, using the same parameters as detailed above. Following electroporation, the cells were transferred to a recovery module (another growth module), allowed to recover in SOC medium containing carbenicillin. After recovery, the cells were held at 4° C. until retrieved, after which an aliquot of cells were plated on LB agar supplemented with chloramphenicol, and kanamycin. To quantify both lacZ and galK edits, replica patch plates were generated on two media types: 1) MacConkey agar base supplemented with lactose (as the sugar substrate), chloramphenicol, and kanamycin, and 2) MacConkey agar base supplemented with galactose (as the sugar substrate), chloramphenicol, and kanamycin. All liquid transfers were performed by the automated liquid handling device of the automated multi-module cell processing system.

In this recursive editing experiment, 41% of the colonies screened had both the lacZ and galK edits, the results of which were comparable to the double editing efficiencies obtained using a “benchtop” or manual approach.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, ¶6.

1. A method for insertion of large DNA sequences into a population of cells and identifying cells with a desired phenotype or genotype comprising the steps of:
(a) designing and synthesizing an editing cassette comprising a large donor DNA sequence, wherein the editing cassette further comprises a gRNA comprising homology to a target sequence in the cells;
(b) inserting the editing cassette into a plasmid backbone resulting in an editing plasmid;
(c) transforming the population of cells with editing plasmid;
(d) allowing editing to take place in the population of cells to produce edited cells, wherein editing comprises integrating the editing plasmid into genomes of the population of cells; and
(e) selecting for edited cells comprising the integrated plasmid by screening for the desired phenotype or genotype.

2. The method of claim 1, wherein the editing cassette or the plasmid backbone further comprises a coding sequence for a nuclease and one or more promoters driving transcription of the coding sequence for the nuclease.

3. The method of claim 2, wherein the nuclease is selected from the group consisting of MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, MAD20, MAD2001, MAD2007, MAD2008, MAD2009, MAD2011, MAD2017, MAD2019, MAD297, MAD298, or MAD299.

4. The method of claim 1, wherein the target sequence is a neutral integration site in the genomes of the cells.

5. The method of claim 1, wherein the editing cassette further comprises a barcode sequence corresponding to the large donor DNA sequence.

6. The method of claim 1, wherein the editing cassette further comprises a selectable marker sequence and one or more promoters driving transcription of the editing cassette and/or the selectable marker sequence, and wherein the method further comprises a selection step between the transforming and allowing steps.

7. The method of claim 1, wherein the editing cassette further comprises an amplification priming site at a 3′ end of the editing cassette.

8. The method of claim 1, wherein the plasmid backbone further comprises a selectable marker sequence and one or more promoters driving transcription of the selectable marker sequence, and wherein the method further comprises a selection step between the transforming and allowing steps.

9. The method of claim 1, wherein the plasmid backbone further comprises a barcode sequence corresponding to the plasmid backbone.

10. The method of claim 1, wherein the large donor DNA sequence is from 100 bp to 100 Kb in length.

11. The method of claim 10, wherein the large donor DNA sequence is from 500 bp to 50 Kb in length.

12. The method of claim 10, wherein the large donor DNA sequence comprises one or more endogenous genes.

13. The method of claim 1, wherein the editing cassette or the plasmid backbone further comprises a landing pad.

14. The method of claim 13, further comprising:
(i) transforming the edited cells with a vector carrying an additional large donor DNA sequence, wherein the vector carrying the additional large donor DNA sequence further comprises a coding sequence for a recombinase or a meganuclease under control of an inducible promoter;
(ii) inducing expression of the recombinase or meganuclease to insert the additional large donor DNA sequence into the landing pad; and
(iii) screening for cells comprising the desired phenotype or genotype.

15. The method of claim 14, wherein the vector carrying the additional large donor DNA sequence further comprise a selectable marker and the method further comprises a selection step between the transforming and inducing steps.

16. The method of claim 14, wherein the vector carrying the additional large donor DNA sequence is another editing plasmid.

17. The method of claim 1, wherein the cells are microbial cells.

18. The method of claim 16, wherein the cells are bacterial cells.

19. The method of claim 18, wherein the cells are Escherichia coli cells.

20. The method of claim 1, wherein the cells are mammalian cells.
